SEQUENCE LISTING (47 sequences)

SEQ ID NO: 1 - protein, 215 aa, Rat
MPAFNRLLPLASLVLIYWVRVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLFIEMIYCYRKVSKAEEAAQENASDYLAIPSENKENSVVPVEE

SEQ ID NO: 2 - protein, 215 aa, Homo sapiens
MPAFNRLFPLASLVLIYWVSVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLLIEMIYCYRKVSKAEEAAQENASDYLAIPSENKENSAVPVEE

SEQ ID NO: 3 - DNA, 2220 bp, rat
cgtggccctggagagggacggttttgaccacctaatcgtccagcatcggggcttcgcaagatccaggaacgcgccccacggaaaggggtccctcggtctacccatcctccacctctgagatcacccaccccaccggaggtcccacctctttccacccctgaaggacctcctgtgagcccgggaccctgtgtacaggactgaagtggaacaaattctgtagcccagacgacggctggagtggggacacgcccaactgaagaagcctgccccgccgtagaagcccgagatcctgagtctcggtggattgaagtcgttgtccctgggggaggcaagagcttcagaaatcgcttacggtggaaaagatgcctgccttcaacagattgcttcccctagcttctctagtgctcatctactgggtcagagtctgcttccctgtgtgtgtggaagtgccctcggagacagaagcggtgcagggcaatcccatgaagctgaggtgcatctcctgcatgaagagggaggaggtggaggccaccactgtggtggagtggttctacaggcctgagggcggtaaagatttccttatatatgagtatcggaatggccaccaggaagtggagagccccttccaaggccgtctgcagtggaatgggagcaaagacctgcaggacgtatccatcactgtactcaatgtcactttgaatgactctggcctctacacatgcaatgtgtccagggagttcgaattcgaggcacacaggccttttgtgaagaccacgagactgatacctttgcgagtcactgaagaggcgggagaagacttcacctccgtggtctcggaaatcatgatgtacatcctcctggtcttcctcaccttgtggctgtttattgagatgatctattgctacagaaaggtctctaaggccgaagaggcagcacaggaaaatgcgtctgactaccttgctatcccttcagagaacaaggagaactctgtggtacctgtggaggaataatgtggtgtgacttgaggtgatgtacacaggcatctgggagggtgatctgagtgctgagggactggatatccccagttcagtgatgccagcaatatcaggaagtgccccaggtgtcccaacacatccatcttttctattcatcaaccaccaacccaatgtgagattttcacctgacttccgaactctatcagaactctacacatctttaccttgcctgaaccgaagagccaacatctatctctacacggactaaacctcactctgttcttgcttccaaccaagtaactcccaacttaactagagttgttccctatgttccaaatgatttagacaagtactggagagtagtattacctctgccctgactgtctgtgactgggtcattctccactgcagcaaaaggatggatataaatcggaagaaagccctgactagtttgtcttaaagccaaagcgtgccacgtacgtactttgattcattgaagtcagtttttcctgcttctcagagcgccagaaagcatgcccctaatgcttgcagggacatcatctgtgtgcactggaacgctttctggagctcagtgtttggaggctgtatccccataatcctgaagacctggagcaaacccagaacttccaggaagtcccaaggaaggatccaggacagtttcagggtctcgaaaatgatataacacactcctgatattggaaacatggatgagtgacctttctggattgaaactcctcagttcttcatgtctcagtgtctgtggatcagtattagtcctcgttttacaggaggaaactgagactcacacaaggctgaacaggacatttaggggattaaactgggccagagatgactttcctgccaccaacctcacactccctgggatgagaggtatttttgaggactctaacattcagcatgccatttgcccagcggaagctgactgccacagatctgaggaactggaaaccaggtaagaaaacacagacggcatgagatagacttcaggatttcacacaaagatttgtgaatctgaagcatcctccaggagagacggcacccgagggcaatatctctgtgatgaaaaatggttttagtctgaaatggacagtcaacagagagacaaagatgggcgtgtagcttctaaatacctcacctgtagatgtcacgtttgggtactggtgtttgtaaagtcccccacctg

SEQ ID NO: 4 - DNA, 1261 bp, Homo sapiens
ccctcccttccgagctgagcttaccctgggcgcaaacgagcgaggcaggggcgcgagtggaagctggagttccggggtgggcggggaggcgactgtccgtggtgctgagcgccggcgagagcgggcgcggagcggctgatcggctccctcgaactggggaggtccagtggggtcgcttagggcccaaagcccccacccggctccaaaagctcccagggcctccccaggcaccggtgctcggcccttccttcggtcagaaagtcgccccctgggggcagttcgtcccaaagggtttcctcgaaagaatctgagagggcgcagtccttgaccgagggaatctctctgtgtagccttggaagccgccagccccagaagatgcctgccttcaatagattgtttcccctggcttctctcgtgcttatctactgggtcagtgtctgcttccctgtgtgtgtggaagtgccctcggagacggaggccgtgcagggcaaccccatgaagctgcgctgcatctcctgcatgaagagagaggaggtggaggccaccacggtggtggaatggttctacaggcccgagggcggtaaagatttccttatttacgagtatcggaatggccaccaggaggtggagagcccctttcaggggcgcctgcagtggaatggcagcaaggacctgcaggacgtgtccatcactgtgctcaacgtcactctgaacgactctggcctctacacctgcaatgtgtcccgggagtttgagtttgaggcgcatcggccctttgtgaagacgacgcggctgatccccctaagagtcaccgaggaggctggagaggacttcacctctgtggtctcagaaatcatgatgtacatccttctggtcttcctcaccctgtggctgctcatcgagatgatatattgctacagaaaggtctcaaaagccgaagaggcagcccaagaaaacgcgtctgactaccttgccatcccatctgagaacaaggagaactctgcggtaccagtggaggaatagaacaggagcagtgtgacatgaggtggcctgaacacctgagggactggacatcccatgttcagcaatgtcaatggcatcaggagggcgccccaagggccccatcgcttcccttcatgcatccattgttctgttcattcattcatccatacatccacctgcctctgagctttcacctctgactccctaactccatcagacctctacgcaccataagactctgccagaactgagaagccgg

SEQ ID NO: 5 - protein, 24 aa, Homo sapiens
MPAFNRLFPLASLVLIYWVSVCFP

SEQ ID NO: 6 - protein, 24 aa, rat
MPAFNRLLPLASLVLIYWVRVCFP

SEQ ID NO: 7 - protein, 19 aa, homo sapiens
MPAFNRLFPLASLVLIYWV

SEQ ID NO: 8 - protein, 19 aa, rat
MPAFNRLLPLASLVLIYWV

SEQ ID NO: 9 - protein, 12 aa, homo sapiens
MPAFNRLFPLAS

SEQ ID NO: 10 - protein, 12 aa, rat
MPAFNRLLPLAS

SEQ ID NO: 11 - protein, 15 aa, homo sapiens
FPLASLVLIYWVSVC

SEQ ID NO: 12 - protein, 15 aa, rat
LPLASLVLIYWVRVC

SEQ ID NO: 13 - protein, 5 aa, homo sapiens
SVCFP

SEQ ID NO: 14 - protein, 5 aa, rat
RVCFP

SEQ ID NO: 15 - protein, 11 aa, Homo sapiens
VLIYWVSVCFP

SEQ ID NO: 16 - protein, 11 aa, rat
VLIYWVRVCFP

SEQ ID NO: 17 - protein, 39 aa, homo sapiens
MPAFNRLFPLASLVLIYWVSVCFPVCVEVPSETEAVQGN

SEQ ID NO: 18 - protein, 39 aa, rat
MPAFNRLLPLASLVLIYWVRVCFPVCVEVPSETEAVQGN

SEQ ID NO: 19 - protein, 9 aa, Homo sapiens
CVEVPSETE

SEQ ID NO: 20 - protein, 17 aa, Homo sapiens
WVSVCFPVCVEVPSETE

SEQ ID NO: 21 - protein, 17 aa, Rat
WVRVCFPVCVEVPSETE

SEQ ID NO: 22 - protein, 159 aa, Homo sapiens
MPAFNRLFPLASLVLIYWVSVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSE

SEQ ID NO: 23 - protein, 159 aa, Rat
MPAFNRLLPLASLVLIYWVRVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSE

SEQ ID NO: 24 - protein, 10 aa, Homo sapiens
TTRLIPLRVT

SEQ ID NO: 25 - protein, 13 aa, Homo sapiens
CMKREEVEATTVV

SEQ ID NO: 26 - protein, 10 aa, Homo sapiens
YEYRNGHQEV

SEQ ID NO: 27 - protein, 12 aa, Homo sapiens
WNGSKDLQDVSI

SEQ ID NO: 28 - protein, 14 aa, Homo sapiens
SREFEFEAHRPFVK

SEQ ID NO: 29 - protein, 9 aa, Homo sapiens
VESPFQGRL

SEQ ID NO: 30 - protein, 13 aa, Homo sapiens
EEAGEDFTSVVSE

SEQ ID NO: 31 - protein, 34 aa, Homo sapiens
RKVSKAEEAAQENASDYLAIPSENKENSAVPVEE

SEQ ID NO: 32 - protein, 7 aa, Homo sapiens
DYLAIPS

SEQ ID NO: 33 - DNA, 22 bp, Artificial sequence
  primer used to amplify nucleic acid sequences encoding b3 subunit nucleic acid of rat or human
atgcctgccttcaacagattgc

SEQ ID NO: 34 - DNA, 20 bp, Artificial sequence
  primer used to amplify nucleic acid sequences encoding b3 subunit nucleic acid of rat or human
ttattcctccacaggtacca

SEQ ID NO: 35 - DNA, 45 bp, Artificial sequence
  antisense radiolabelled oligonucleotide probe used in the in situ hybridization experiments
ggggaagcaatctgttgaaggcaggcatcttttccaccgtaagcg

SEQ ID NO: 36 - DNA, 18 bp, Artificial sequence
  primer used to amplify nucleic acid encoding a b3 sub-unit from a voltage-gated sodium channel
ggtgaagcaatatggccg

SEQ ID NO: 37 - DNA, 18 bp, Artificial sequence
  reverse primer (nucleotides 1317-1300) corresponding to unique sequence in the 3′ untranslated region of each b subunit
agatgaggcccagaaccc

SEQ ID NO: 38 - DNA, 20 bp, Artificial sequence
  forward primer (nucleotides 1942-1961) corresponding to unique sequence in the 3′ untranslated region of each b subunit
ggaagctgactgccacagat

SEQ ID NO: 39 - DNA, 20 bp, Artificial sequence
  reverse primer (nucleotides 2209-2190) corresponding to unique sequence in the 3′ untranslated region of each b subunit
cctgggggactttacaaaca

SEQ ID NO: 40 - DNA, 19 bp, Artificial sequence
  a-tubulin forward primer (nucleotides 298-316) corresponding to unique sequence in the 3′ untranslated region of each b subunit
cactggtacgtgggtgagg

SEQ ID NO: 41 - DNA, 22 bp, Artificial sequence
  reverse primer (nucleotides 469-448) corresponding to unique sequence in the 3′ untranslated region of each b subunit
tttgacatgatacagggactgc

SEQ ID NO: 42 - DNA, 44 bp, Artificial sequence
  rat b1 (nucleotides 1296-1252) primer used in immobilization of nucleic acid probe on a substrate
gcttgatggggtgaagaggggtcgggacagggacagtagtgggc

SEQ ID NO: 43 - DNA, 45 bp, Artificial sequence
  rat a IIA (nucleotides 1659-1615) primer used in immobilization of nucleic acid probe on a substrate
gcagaatccagagacttcagcggggcaggcgggataggtgttttc

SEQ ID NO: 44 - protein, 218 aa, Rat
MGTLLALVVGAVLVSSAWGGCVEVDSETEAVYGMTFKILCISCKRRSETTAETFTEWTFRQKGTEEFVKILRYENEVLQLEEDERFEGRVVWNGSRGTKDLQDLSIFITNVTYNHSGDYECHVYRLLFFDNYEHNTSVVKKIHLEVVDKANRDMASIVSEIMMYVLIVVLTIWLVAEMVYCYKKIAAATEAAAQENASEYLAITSESKENCTGVQVAE

SEQ ID NO: 45 - protein, 119 aa, Rat
IVVYTDREVYGAVGSQVTLHCSFWSSEWVSDDISFTWRYQPEGGRDAISIFHYAKGQPYIDEVGTFKERIQWVGDPSWKDGSIVIHNLDYSDNGTFTCDVKNPPDIVGKTSQVTLYVFE

SEQ ID NO: 46 - protein, 8 aa, Homo sapiens
EGGKDFLI

SEQ ID NO: 47 - protein, 34 aa, Rat
RKVSKAEEAAQENASDYLAIPSENKENSVVPVEE

(19) United States
(12) Patent Application Publication
(10) Pub. No.: US 2004/0248240 A1
(43) Pub. Date: Dec. 9, 2004

(54) Novel family of beta sub-unit proteins from a voltage-gated sodium channel, nucleic acids encoding them and therapeutic or diagnostic uses thereof

(76) Inventors: Peter Cox, Ramsgate (GB); Alistair Dixon, Cambridge (GB); Antony Jackson, Cambridge (GB); Kevin Morgan, Edinburgh (GB)

Correspondence Address: Thomas J. Kowalski, c/o FROMMER LAWRENCE & IIAUG LLP, 745 Fifth Avenue, New York, NY, 10151, US

(21) Appl. No.: 09/977,579

(22) Filed: Oct. 15, 2001

Related U.S. Application Data
(63) Continuation-in-part of application No. PCT/EP00/01783, filed on Feb. 24, 2000.
(60) Provisional application No. 60/129,473, filed on Apr. 15, 1999.

(51) Int. Cl. C07K 14/705; C07H 21/04

(52) U.S. Cl. 435/69.1; 435/320.1; 435/325; 530/350; 536/23.5

(57) ABSTRACT

The present invention relates to a novel family of beta sub-unit proteins from a voltage-gated sodium channel, and particularly the human and the rat beta sub-units which have been collectively identified as β3, in view of their close structural relationship.

The invention also deals with the use of the β3 sub-unit polypeptide or a fragment thereof as well as of the nucleic acids encoding same for therapeutic, diagnostic and screening purposes.

### RELATED APPLICATIONS/INCORPORATION BY REFERENCE

This application is a continuation-in-part of PCT/EP00/01783, filed Feb. 24, 2000, designating the U.S., published Oct. 26, 2000 as WO 00/63367, and claiming priority from U.S. Provisional Application No. 60/129,473, filed Apr. 15, 1999. The foregoing applications, and more generally all documents cited herein (individually and collectively “application documents”), and all documents cited or referenced in the application documents (including documents cited during any prosecution of any patent applications, publications or patents), including any manufacturer's specifications, data sheets and the like for any commercially available products mentioned herein, are hereby incorporated herein by reference.

### FIELD OF THE INVENTION

The present invention relates to a novel family of beta sub-unit proteins from a voltage-gated sodium channel, and particularly the human and the rat beta sub-units.

The invention also deals with the use of the β3 sub-unit polypeptide or a fragment thereof as well as of the nucleic acids encoding sane for therapeutic, diagnostic and screening purposes.

### BACKGROUND OF THE INVENTION

Sodium channels play a central role in physiology. They transmit depolarising impulses rapidly throughout cells and cell networks, thereby enabling co-ordination of higher processes from cognition to locomotion. The ion permeability and voltage sensing is primarily determined by the alpha sub-unit of the sodium channel complex as this forms the pore. There are at least two major classes and at least eight genes encoding sodium channels.

Voltage-dependent Na⁺ channels have long been recognised as targets for anti-arrhythmic and local anaesthetic drugs. Since the mid-1980s, Na⁺ channels have become widely accepted as the primary target of anticonvulsants with pharmacological profiles similar to those of phenytoin, carbamazepine, and lamotrigine.

Alteration of ion channel function is an important pathophysiological mechanism of various familial muscle diseases. Na⁺ channel mutations underlie the aberrant excitability characteristic of some skeletal muscle myotonias and paralysis, as well as chromosome 3-linked long-QT syndrome, an inherited cardiac arrhythmia. In general, these mutations disable inactivation of the Na⁺ channel, producing either repetitive action potential firing (myotonia) or electrical silence (flaccid paralysis) in skeletal muscles. A similar defect in the cardiac Na⁺ channel produces action potential prolongation and a predisposition to repetitive electrical activity in the heart leading to polymorphic ventricular tachycardia.

Additional determinants of sodium channel function are the presence or absence of auxiliary β1 and 2 sub-units. These are important modulators of Na⁺ channel function. Biochemical studies first revealed the existence of two distinct sub-units (β1 and β2) associated with the brain Na⁺ channel. Antibodies directed to the α or β sub-unit appeared to immunoprecipitate the entire brain Na⁺ channel complex with a sub-unit stoichiometry of 1α:1β1:1β2. The β1 sub-unit is non-covalently associated, while β2 is linked by a disulphide bond to the α sub-unit. The β1 and β2 sub-units have been cloned and the deduced primary structures indicate that they are unrelated proteins of molecular weights of 23 and 21 kDa, respectively. The predicted transmembrane topology of the sub-units is similar: each contains a small carboxy-terminal cytoplasmic domain, a single membrane-spanning segment, and a large amino-terminal extracellular domain with several consensus sites for N-linked glycosylation.

Expression of β2 with neuronal sub-units in Xenopus oocytes increased the current amplitude, modulated gating and increased the membrane capacitance. Co-expression of β1 sub-units with either neuronal or skeletal muscle sub-units in oocytes also produced clear-cut effects on channel function. The current density increased, activation and inactivation gating were accelerated, and the steady-state inactivation curves were shifted in the hyperpolarizing direction. The mRNA encoding the β1 sub-unit appears to be widely expressed and clearly forms an important component of neuronal and skeletal muscle Na⁺ channels. It has recently been established that β1 sub-units modify the interactions of neurotoxins and local anaesthetics with the rat brain α sodium channel.

Until recently there was no known linkage of a phenotype with the β sub-units. However, a mutation in the β1 sub-unit gene SCNIB has been shown to be associated with Febrile seizures and generalised epilepsy.

All the known sub-units of the Na⁺ channel are modified by glycosylation. The β1, β2 and brain and muscle sub-units are heavily glycosylated, with up to 40% of the mass being carbohydrate. In contrast, the cardiac sub-unit contains only 5% of sugar by weight. Sialic acid is a prominent component of the N-linked carbohydrate of the Na⁺ channel. The addition of such a highly charged carbohydrate has predictable effects on the voltage dependence of gating through alteration of the surface charge of the channel protein. Neuraminidase treatment to remove sialic acid from expressed skeletal muscle channels produces a depolarizing shift of steady-state inactivation. It has also been shown that co-translational glycosylation is essential for the maintenance of cell surface expression of the Na⁺ channel in neurones and Schwann cells. Inhibition of glycosylation by tunicamycin reversibly decreases the number of STX binding sites on neuroblastoma cells. Tunicamysin also inhibits palpitation, sulphation and disulphide attachment of the β2 sub-unit, preventing the assembly of functional Na⁺ channels.

The modulation of the voltage gated sodium channel was thought to be through the β1 sub-unit. However, analysis of the distribution of these sub-units in human and rat indicates an asymmetric distribution. This poses the question as to what other mechanisms are employed to impart correct function to the voltage gated sodium channel.

The inventors have discovered a distinct second auxiliary regulatory sub-unit β3 which could at least partially explain this discrepancy.

### SUMMARY OF THE INVENTION

The invention relates to a purified or isolated nucleic acid encoding a β3 sub-unit from a voltage-gated sodium channel or a sequence complementary thereto.

The invention also concerns a β3 sub-unit polypeptide or a peptide fragment thereof as well as antibodies specifically directed against such β3 sub-unit polypeptide or peptide fragment.

Oligonucleotide probes or primers specifically hybridizing to a nucleic acid encoding a β3 sub-unit or to a sequence complementary thereof are also part of the invention as well as DNA amplification and detection methods using said primers and probes.

A further object of the invention consists of recombinant vectors comprising any of the nucleic acid sequence described herein, and in particular recombinant vectors comprising a nucleic acid sequence encoding a β3 sub-unit of the invention, the invention also encompasses host cells and transgenic non-human mammals comprising said nucleic acid sequences or recombinant vectors.

The present invention is also directed to a method of screening for agonist and antagonist molecules or substances of sodium channels as well as to gene therapy methods involving selective addition or removal of the β3 sub-unit nucleic acid sequence in a genome, particularly via an anti-sense technology.

The invention also pertains to methods for the diagnosis of diseases states involving a dysfunction of a voltage-gated sodium channel through analysis, with either the oligonucleotides or antibodies of the invention, of the expression of the β3 sub-unit. Conditions which involve the β3 sub-unit include pain, epilepsy, stroke, ischemia and heart disease. Furthermore, the human β33 gene has been mapped to chromosome 11q 23.3. The genes for β2 and the human N-CAM gene, which contains a V-type Ig domain, are also localised close to this region (Eubanks, J et al, 1997 & Nguyen, C. et al, 1986). Diseases which map to 11q 23.3 include Jacobsen Syndrome, Familial Nonchromaffin Paraganglioma, Phenylketonuria due to PTS deficiency and Charcot Marie Tooth disease. Therefore, characterisation of the modulation of the expression of β3 could be useful for the diagnosis of these diseases.

The invention will be described hereinafter more in details and will be illustrated by the following figures:

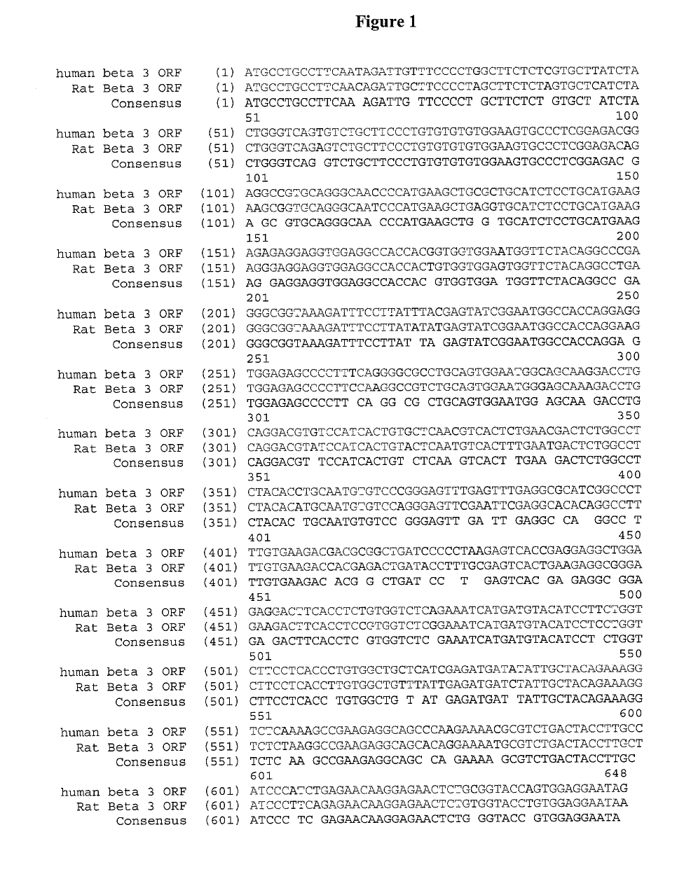

FIG. 1: Sequence alignment of the human and rat β3 sub-unit coding sequences.

Upper line: coding sequence of the human β3 sub-unit.

Middle line: coding sequence of the rat β3 sub-unit.

Lower line: consensus sequence containing the nucleotides that are common to both the human and rat β3 sub-units.

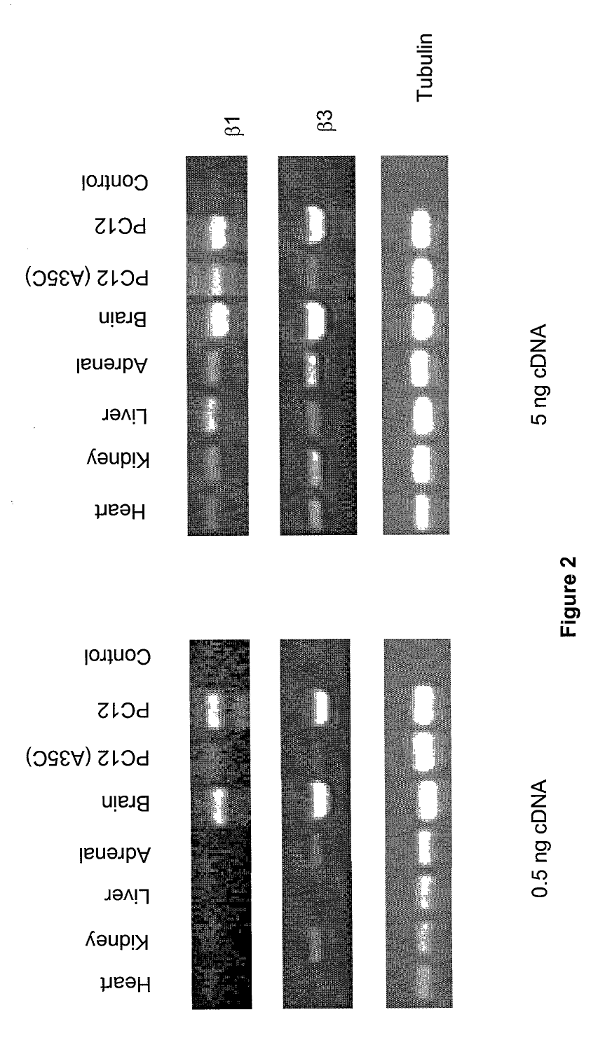

FIG. 2: Tissue expression of β3 and β1 subunits in rat by Polymerase Chain Reaction.

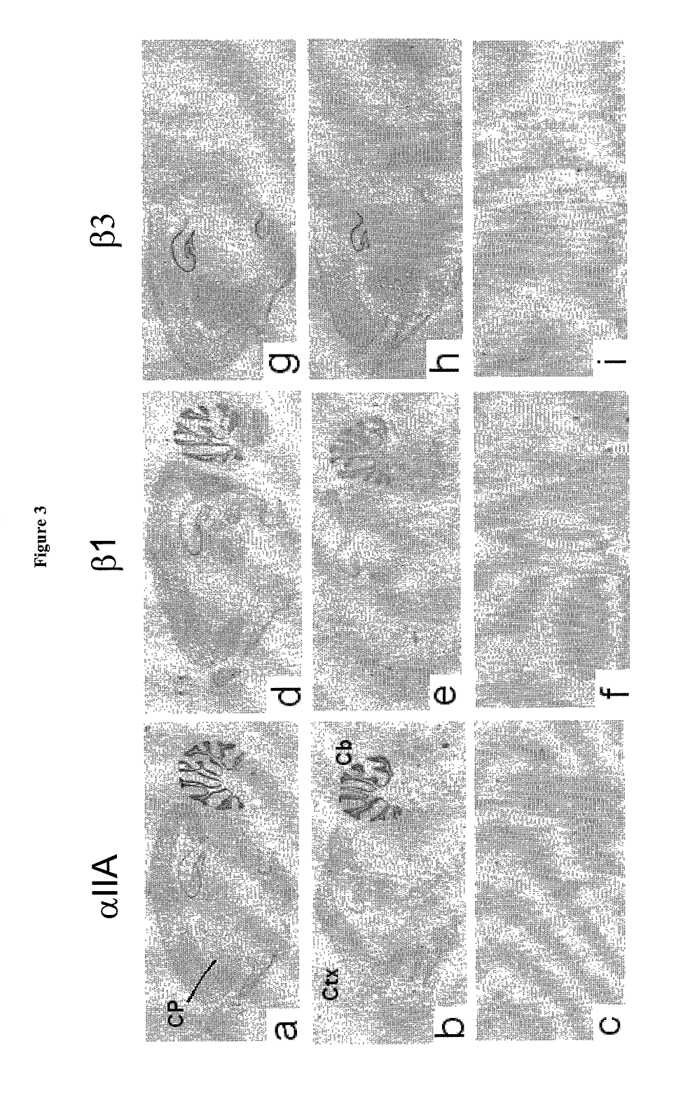

FIG. 3: In situ distribution of sodium channel subunits in adult rat brain. X-ray autoradiographs of separate sagittal sections of rat brain (taken from the same animal) showing the distribution of rat αIIA (a,b,c); rat β1 (d,e,f) and rat β3 (g,h,i) mRNA transcripts as revealed by in situ hybridisation with specific olignucleotide probes. Control reactions with 100-fold excess unlabelled probes are shown for αIIA(c);β1(f) and β3 (i). Slides were exposed to X-ray film for 10 days. Dark areas indicate high expression levels. Cb, cerebellum; Ctx, cortex; CP, caudate putamen.

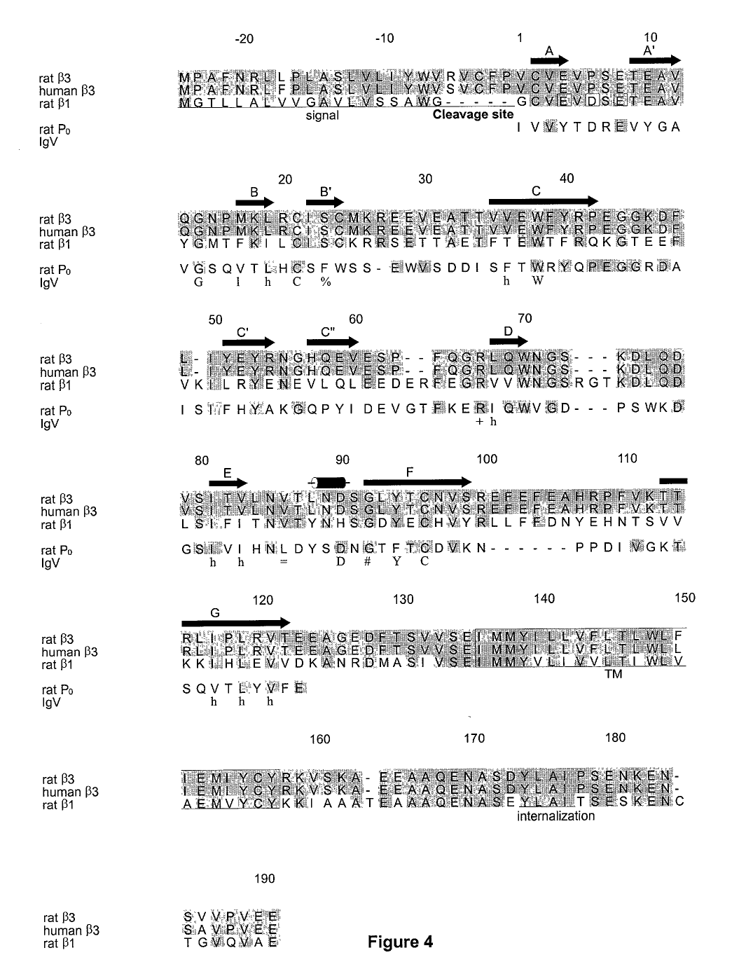

FIG. 4: Amino acid sequence alignment of the human and rat β3, rat β1 sub-unit and rat myelin P0 protein.

Line-1: Amino acid sequence of rat β3

Line-2: Amino acid sequence of human β3

Line-3: Amino acid sequence of rat β1

Line-4: Amino acid sequence of rat myelin P0

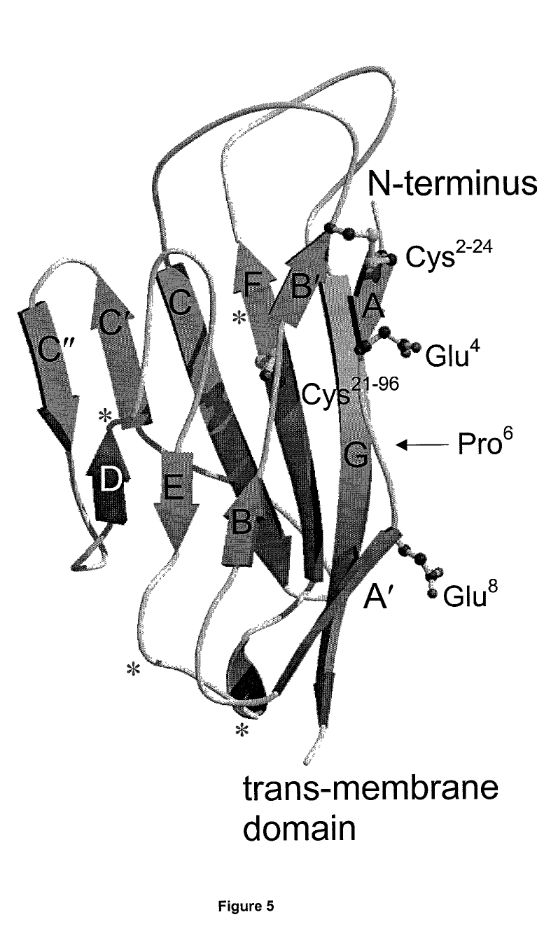

FIG. 5: Three dimensional structure of the extra-cellular domain of the β3 sub-unit.

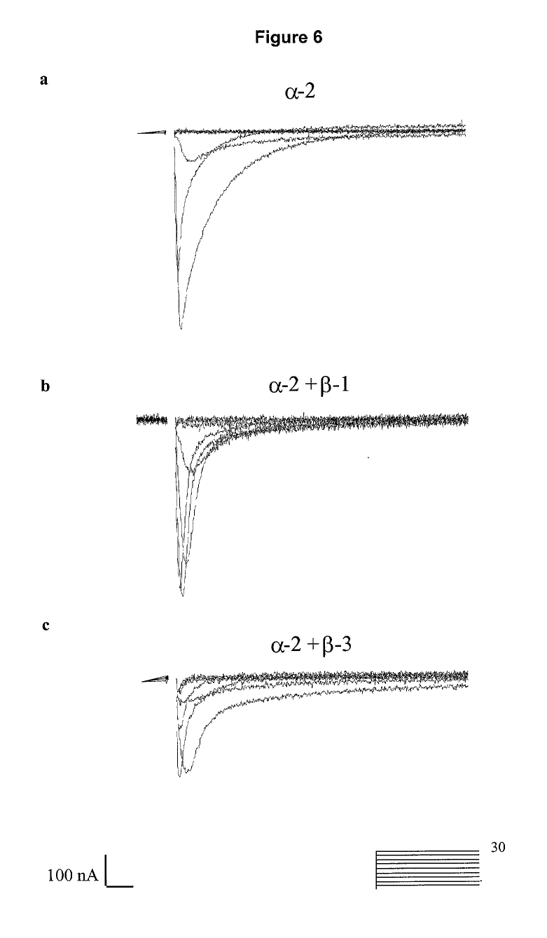

FIG. 6: Na⁺ current curves in oocytes expressing either the IIA α sub-unit alone, or IIA α and β1 or β3 sub-units. Inward Na⁺ currents were evoked by applying 5 mV depolarizing pulses from a holding potential of −100 mV, from −80 mV to +30 mV. a. Na⁺ currents recorded from oocytes expressing IIA α subunit alone. Inactivation at −10 mV was best-fitted with a double exponential function, where τ₁=2±0.3 ms and τ₂=12.7±2.4 ms (n=4). b. Na⁺ currents recorded from oocytes coexpressing IIA α and β₁ subunits. Inactivation was best-fitted with a double exponential function, where τ₁=1.3±0.3 ms and τ₂=22.7±7.7 ms at −10 mV (n=4). c. Na⁺ currents recorded from oocytes coexpressing IIA₂ α and the β₃ subunits. Inactivation was best-fitted with a double exponential function, where τ₁=1±0.1 ms and τ₂=23.8±6.3 ms at −10 mV (n=4).

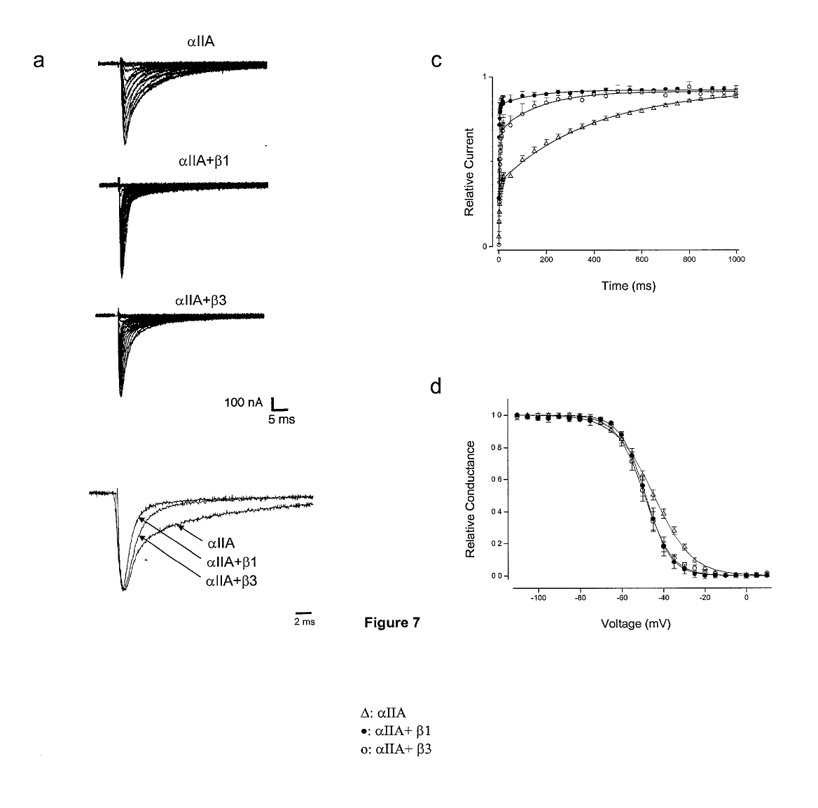

FIG. 7: Na⁺ current curves in oocytes expressing either the IIA α sub-unit alone, or IIA α and β1 or β3 sub-units.

### DETAILED DESCRIPTION OF THE INVENTION

The inventors have found a novel family of beta sub-unit proteins that cooperate with at least one α sub-unit of voltage-gated sodium channels to form an active sodium channel. This novel beta sub-unit family has been termed β3 and can be identified as such through common structural sequence features, such as a high homology within the sequences that will be described hereafter.

The inventors have found novel nucleic acid sequences encoding a β3 sub-unit from a voltage-gated sodium channel. They have shown that this β3 sub-unit was biologically functional and that co-expression of the β3 sub-unit with an α2 sub-unit from a voltage-gated sodium channel significantly increases the rate of inactivation of the channel, as compared with the expression of the α2 sub-unit alone. Moreover, co-expression of the β3 sub-unit of the invention with an α2 sub-unit increases the rate of recovery from inactivation of the sodium channel as compared with the expression of the α2 sub-unit alone.

The inventors have thus demonstrated that the β3 sub-unit of the invention is involved in the regulation of the sodium currents induced by the voltage-gated sodium channels. They have also determined that the β3 sub-units of the invention may be valuable targets for drugs capable of up regulating or down regulating the activity of voltage-gated sodium channels, in particular drugs designed for preventing or treating pain, epilepsy (typically febrile seizures and generalized epilepsy), stroke, ischemia, heart disease, Jacobsen Syndrome, Familial Nonchromaffin Paraganglioma, Phenylketonuria due to PTS deficiency and Charcot Marie Tooth disease. Appropriate modulation of β3 may therefore be taken into account in the treatment of such diseases.

In another aspect of the present invention, the nucleic acids encoding the β3 sub-unit may be used to design polynucleotides that can interfere with the functional expression of the β3 sub-unit both in vitro and in vivo and hence also be useful in the treatment of diseases set forth above.

Nucleotide Sequences Encoding β3

A first object of the present invention consists of a purified or isolated nucleic acid encoding a β3 sub-unit from a voltage-gated sodium channel, or a sequence complementary thereto. Preferred nucleic acids encoding a β3 sub-unit include those isolated from rat and human brain, preferably, those of SEQ ID N°3 and SEQ ID N°4.

Using total mRNA rom wild type PC12 and variant PC12 cell lines, the inventors have isolated the cDNA encoding the rat β3 sub-unit. From the rat β3 sub-unit cDNA sequence information, the inventors have also isolated and cloned the human cDNA encoding the human β3 sub-unit.

As shown in FIG. 1, the coding sequences (ORF) of the rat and human β3 sub-units are highly homologous, with only 70 non identical nucleotides out of a total length of 648 nucleotides (>89% nucleotide identity between the two coding sequences). Hence, the inventors believe that nucleic acids encoding β3 in other mammalian species will share a strong nucleotide identity with the corresponding rat and human nucleotide sequences.

Consequently, a further object of the invention consists of a purified or isolated nucleic acid having at least 90%, preferably 95%, more preferably 98%, and most preferably 99% nucleotide identity with the nucleotides sequence of SEQ ID N°3, or a sequence complementary thereto.

The invention also deals with a purified or isolated nucleic acid comprising a sequence encoding the Open Reading Frame (ORF) of a β3 sub-unit from a voltage-gated channel present in the rat brain, such sequence having at least 90%, preferably 95%, more preferably 98% and most preferably 99% with the polynucleotide beginning at the nucleotide located in position 363 and ending at the nucleotide located in position 1010 of the nucleotide sequence of SEQ ID N°3.

The invention relates also to a purified or isolated nucleic acid having at least 90%, preferably 95%, more preferably 98%, and most preferably 99% nucleotide identity with the nucleotide sequence of SEQ ID N°4, or a sequence complementary thereto.

The invention is also directed to a purified or isolated nucleic acid comprising a sequence encoding the Open Reading Frame (ORF) of a β3 sub-unit from a voltage-gated channel present in the human brain, such sequence having at least 90%, preferably 95%, more preferably 98% and most preferably 99% with the polynucleotide beginning at the nucleotide located in position 376 and ending at the nucleotide located in position 1023 of the nucleotide sequence of SEQ ID N°4.

Another object of the invention consists of a purified or isolated nucleic acid encoding a polypeptide having at least 80%, preferably 90%, more preferably 95%, and most preferably 98% aminoacid identity with the rat polypeptide of the aminoacid sequence of SEQ ID N°1 or with a peptide fragment thereof, or a sequence complementary thereto.

The invention further concerns a purified or isolated nucleic acid encoding a polypeptide having at least 80%, preferably 90%, more preferably 95%, and most preferably 98% aminoacid identity with the human polypeptide of the aminoacid sequence of SEQ ID N°2 or with a peptide fragment thereof or a sequence complementary thereto.

The term “isolated” requires that the material be removed from its original environment (e.g. the natural environment if it is naturally occurring). For example, a naturally occurring polynucleotide or a peptide present in a living animal is not isolated, but the same polynucleotide or peptide, separated from some or all of the coexisting materials in the natural system, is isolated.

Such polynucleotide can be part of a vector and/or such polynucleotide or peptide can be part of a composition, and still be isolated in that the vector or composition is not a part of its natural environment.

The term “purified” does not require absolute purity; rather, it is intended as a relative definition.

Purification of starting materials or natural materials to at least one order of magnitude, preferably two or three orders, and more preferably four or five orders of magnitude is expressly contemplated.

Throughout the present specification, the expression “nucleotide sequence” may be employed to designate indifferently a polynucleotide or a nucleic acid. More precisely, the expression “nucleotide sequence” encompasses the nucleic material itself and is not restricted to the sequence information (i.e. the succession of letters chosen among the four base letters) that biochemically characterizes a specific DNA or RNA molecule.

As used interchangeably herein, the term “oligonucleotides”, “nucleic acids” and “polynucleotides” include RNA, DNA, or RNA/DNA hybrid sequences of more than one nucleotide in either single chain or duplex form.

Further to its general meaning understood by the one skilled in the art, the term “nucleotide” is also used herein to encompass modified nucleotides which comprise at least one of the following modifications: (a) an alternative linking group, (b) an analogous form of purine, (c) an analogous form of pyrimidine, or (d) an analogous sugar. For examples of analogous linking groups, purines, pyrimidines, and sugars, see for example PCT publication No WO 95/04064.

The polynucleotide sequences of the invention may be prepared by any known method, including synthetic, recombinant, or a combination thereof as well as through any purification methods known in the art.

β3 Nucleotide Fragments and Assays

The invention also encompasses polynucleotide fragments of a nucleic acid encoding a β3 sub-unit of a voltage-gated sodium channel as described herein, that may be useful either to express a peptide fragment, preferably a biologically active peptide fragment, of this β3 sub-unit, as nucleic acid primers or probes for amplification or detection purposes, or as antisense nucleotides able to regulate the expression of the corresponding gene.

Consequently, the present invention also concerns a purified or isolated polynucleotide comprising at least 10 consecutive nucleotides of a nucleic acid encoding a β3 sub-unit from a voltage-gated sodium channel described herein, preferably at least 10 consecutive nucleotides of any one of the nucleotide sequences of SEQ ID N°3 or 4, or a sequence complementary thereto.

Preferred polynucleotides include those encoding peptides selected from SEQ ID N° 5 to 32 and SEQ ID 46 and 47. Other preferred polynucleotides include those of SEQ ID N° 35 to 43.

The nucleic acids described above consist of a contiguous span which ranges in length from about 10, 12, 15, 18 or 20 to about 25, 35, 40, 50, 70, 80, 100, 250, 500 or 1000 nucleotides, or be specified as being 10, 12, 15, 18, 20, 25, 35, 40, 50, 100, 200, 250, 500 or 1000 nucleotides in length.

These nucleic acids are useful as probes in order to detect the presence of at least a copy of a nucleotide sequence encoding a β3 sub-unit from a voltage-gated sodium channel, more particularly the presence of at least a copy of a nucleotide sequence of SEQ ID N°3 or SEQ ID N°4 or a sequence complementary thereto or a fragment or a variant thereof in a sample. They can also be used to express a selected peptide of the invention.

The nucleic acid probes of the invention may also be used for the analysis of the expression levels and patterns of the β3 sub-unit, such as described in the PCT Application No WO 97/05 277, the entire contents of which is herein incorporated by reference.

Quantitative analysis of the β3 sub-unit expression may also be performed using assays, i.e. a substrate on which has been bound a plurality of nucleic acid probes according to the invention, these probes being either randomly distributed on the substrate or arranged following a one dimensional, two dimensional or multidimensional arrangement. Such assays may additionally comprise nucleic acid probes that do not hybridize with a β3 sub-unit DNA or RNA, such as for example probes specific for α2, β1 or β2 sodium channel sub-unit RNA or DNA sequences. Suitable techniques are, for example, those described by Schena et al (1995; 1996), and also by Sosnowsky et al., (1997), the disclosures of which are herein incorporated by reference.

The invention further deals with a purified or isolated nucleic acid that hybridizes, under stringent hybridization conditions, with a nucleic acid encoding a β3 sub-unit from a voltage-gated sodium channel, or a sequence complementary thereto.

As an illustrative embodiment, stringent hybridization conditions can be defined as follows:

The hybridization step is conducted at 65° C. in the presence of 6×SSC buffer, 5×Denhardt's solution, 0.5% SDS and 100 μg/ml of salmon sperm DNA.

The hybridization step is followed by four washing steps:

two washings during 5 minutes, preferably at 65° C. in a 2×SSC and 0.1% SDS buffer;

one washing during 30 minutes, preferably at 65° C. in a 2×SSC and 0.1% SDS buffer;

one washing during 10 minutes, preferably at 35° C. in a 0.1×SSC and 0.1% SDS buffer,

it being understood that the hybridization conditions defined above are suitable for nucleic acids of approximately twenty nucleotides in length and that these conditions may be also adapted for shorter or longer nucleic acids, according to techniques well known in the art, for example those described by Sambrook et al. (1989).

The appropriate length for probes under a particular set of assay conditions may be empirically determined by the one skilled in the art. The probes can be prepared by any suitable method, including, for example, cloning and restriction of appropriate sequences and direct chemical synthesis by a method such as the phosphodiester method of Narang et al. (1979), the phosphodiester method of Brown et al., (1979), the diethylphosphoramidite method of Beaucage et al. (1981) and the solid support method described in the application N°EP-0 707 792. The disclosures of all these documents are incorporated herein by reference.

Any of the nucleic acids of the present invention can be labelled, if desired, by incorporating a label detectable by spectroscopic, photochemical, biochemical, immunochemical, or chemical means.

For example, useful labels include radio-active substances (³²P, ³⁵S, ³H, ¹²⁵I), fluorescent dyes (5-bromodesoxyuridin, fluorecein, acetylaminofluoren, digoxygenin) or biotin. Examples of non-radioactive labelling of nucleic acid fragments are described in French Patent N°FR-78 10975 or by Urdea et al. (1988) or Sanchez-Pescador et al. (1988).

Advantageously, the probes according to the present invention may have structure and characteristics such that they allow signal amplification, such structural characteristics being, for example, branched DNA probes as those described by Urdea et al. (1991).

Any of the nucleic acid probes of the invention can be conveniently immobilized on a solid support. Solid supports are known those skilled in the art and include the walls of wells of a reaction tray, test tubes, polystyrene beads, magnetic beads, nitro-cellulose strips, membranes, microparticules such as latex particles, sheep red blood cells, duracytes and others.

The nucleic acids of the invention and particularly the nucleotide probes described above can thus be attached to or immobilized on a solid support individually or in groups of at least 2, 5, 8, 10, 12, 15, 20 or 25 distinct nucleic acids of the invention to a single solid support.

In a specific embodiment of a support on which nucleic acid probes of the invention are immobilized, such a support may also contain other immobilized probes, preferably probes that hybridize specifically with a nucleic acid encoding a sub-unit from a voltage-gated sodium channel, or a variant thereof, or a sequence complementary thereto, and more preferably with a nucleic acid encoding an α sub-unit, most preferably an α2 sub-unit of a voltage-gated sodium channel.

The invention also encompasses nucleic acid probes comprising a nucleotide sequence included in any one of the sequences of SEQ ID N°3 and 4 as well as the preferred polynucleotides referred to above wherein at least one nucleotide substitution has been made in order to create a mismatch between this probe and the complementary nucleotide sequence included in any one of SEQ ID N° 3 and SEQ ID No4 to be detected. Under suitable hybridization conditions, these probes will not hybridize anymore with anyone of the nucleotide sequences of SEQ ID N°3 and SEQ ID N°4 or a fragment thereof or a sequence complementary thereto, but will hybridize only with nucleotide sequences which are exactly complementary to the polynucleotide comprised in these probes.

This specific embodiment of the nucleic acid probes of the invention may allow the detection of nucleotide polymorphisms within a nucleic acid sequence encoding a β3 sub-unit of voltage-gated sodium channel, more specifically in a nucleic acid encoding a β3 sub-unit voltage-gated sodium channel from human or rat, and more preferably a nucleic acid of any one of the sequences SEQ ID N°3 and SEQ ID N°4, or a sequence complementary thereto.

Such probes can allow the one skilled in the art to detect mutations occurring in a nucleic acid encoding a β3 sub-unit of the invention, more preferably a nucleic acid encoding a β3 sub-unit from rat of human, and most preferably a nucleic acid or any one of SEQ ID N°3 and SEQ ID N°4.

The invention also deals with a method for detecting the presence of a nucleic acid encoding a β3 sub-unit from a voltage-gated sodium channel, a fragment or a variant thereof or a complementary sequence thereto in a sample, said method comprising the following steps:

(a) bringing into contact a nucleic acid probe or a plurality of nucleic acid probes of the invention which can hybridize with a nucleotide sequence included in a nucleic acid encoding a β3 sub-unit from a voltage-gated sodium channel, or a fragment a variant thereof or a complementary sequence thereto, and a sample to be assayed;

(b) detecting the hybrid complex formed between the probe or the plurality of probes and a nucleic acid in the sample.

In a first preferred embodiment, the nucleic acid encoding a β3 sub-unit from a voltage-gated sodium channel to be detected is preferably a rat or human β3 sub-unit, and more preferably a nucleic acid selected from the group consisting of the nucleotide sequences of SEQ ID N°3 and SEQ ID N°4.

In a second preferred embodiment of this detection method, the nucleic acid probe or the plurality of nucleic acid probes are labelled with a detectable molecule.

In a third preferred embodiment of the method, the nucleic acid probe or the plurality of nucleic acid probes are immobilized on a substrate.

In a fourth preferred embodiment of the method, the nucleic acid contained in the sample is made available to hybridization before step (a), by any conventional procedure well known from the one skilled in the art.

The invention further concerns a kit for detecting the presence of a nucleic acid encoding a β3 sub-unit from a voltage-gated sodium channel, a fragment or a variant thereof or a complementary sequence thereto in a sample, wherein said kit comprises:

(a) a nucleic acid probe or a plurality of a nucleic acid probes as described above;

(b) optionally, a reagent necessary for performing the hybridization reaction.

In a first preferred embodiment of the detection kit, the nucleic acid to be detected encodes a human or rat β3 sub-unit, and consists preferably of any one of the nucleotide sequences of SEQ ID N°3 and SEQ ID N°4, a fragment or a variant thereof, or a sequence complementary thereto.

In a second preferred embodiment of the detection kit, the nucleic acid probe or the plurality of nucleic acid probes are labelled with a detectable molecule.

In a third preferred embodiment of the detection kit, the nucleic acid probe or the plurality of nucleic acid probes are immobilized on a substrate.

The invention is also directed to a polynucleotide primer hybridizing, under the stringent hybridization conditions described herein, with a nucleic acid encoding a β3 sub-unit from a voltage-gated sodium channel of the invention, preferably a rat or a human β3 sub-unit, and more preferably a nucleotide sequence selected from the group consisting of SEQ ID N°3 and SEQ ID N°4.

As an illustrative example, primers according to the present invention may comprise, or may consist of a polynucleotide selected from the group consisting of the nucleotide sequences of SEQ ID N°3, SEQ ID N°34, SEQ ID N° 35, SEQ ID No 42 and SEQ ID N° 43. The use of a pair of primers, for example respectively comprising, or consisting of the nucleotide sequences of SEQ ID N°33 and SEQ ID No34 allows the one skilled in the art to amplify the whole nucleic acid sequences encoding either the human β3 sub-unit or the rat β3 sub-unit of the invention.

In a specific embodiment of a primer according to the invention, such a primer may comprise a 3′ end nucleotide which is not exactly complementary to a target sequence included in anyone of the nucleotide sequences of SEQ ID N°3 or SEQ ID N°4, or a sequence complementary thereto.

According to this specific embodiment of a primer according to the invention, such a primer comprises a 3′ end nucleotide chosen in such a way as to allow hybridization of the primer, and thus the possibility of further elongation, only when a given variant of a β3 sub-unit of the invention is present in the sample containing the target sequence to be amplified, this variant β3 sub-unit nucleic acid sequence corresponding to a genome polymorphism. Particularly, preferred primers encompassed in this specific embodiment will then exclusively hybridize with a given variant of a β3 sub-unit of the invention, and more preferably with a β3 sub-unit of the invention for which a linkage with a detectable phenotype, caused by a disfunction in a voltage-gated sodium channel, and more preferably with pain, epilepsy, stroke and ischemia, heart disease, Jacobsen Syndrome, Familial Nonchromaffin Paraganglioma, Phenylketonuria due to PTS deficiency and Charcot Marie Tooth disease has been shown.

β3 Polypeptides

Another object of the present invention consists of a purified or isolated polypeptide comprising the aminoacid sequence of the β3 sub-unit from a voltage-gated sodium channel, or a peptide fragment or a variant thereof.

In a first embodiment, the polypeptide comprises the aminoacid sequence of the β3 sub-unit from a voltage-gated sodium channel present in the rat brain or a peptide fragment or a variant thereof. A particularly preferred polypeptide is the polypeptide of SEQ ID N°1

In a second preferred embodiment, the polypeptide comprises the aminoacid sequence of the β3 sub-unit or from a voltage-gated sodium channel present in the human brain, or a peptide fragment or variant thereof. A particularly preferred polypeptide is the polypeptide of SEQ ID N°2.

As shown in FIG. 4, the amino acid sequences of the rat and human β3 sub-units have a very strong sequence similarity, with only three non identical aninoacids out of a total length of 191 amino acids when the signal sequence is included (>98% aminoacid identity between the two polypeptides). The sequence similarity between the two polypeptides is even higher (>99% aminoacid identity) if the 24 aminoacid sequence of the signal peptide is not included in the analysis. Thus, the inventors also believe that the β3 polypeptide in other mammalian species will share a strong amino acid identity with the corresponding rat and human amino acid sequences.

Hence, the present invention concerns a polypeptide comprising an aminoacid sequence having at least 90%, preferably 95%, more preferably 98%, and more preferably 99% amino acid identity with the aminoacid sequence of SEQ ID No1 or a peptide fragment thereof.

Also within the scope of the present invention is a polypeptide comprising an aminoacid sequence having at least 90%, preferably 95%, more preferably 98%, and most preferably 99% aminoacid identity with the aminoacid sequence of SEQ ID N°2, or a peptide fragment thereof.

β3 Peptide Fragments

The invention also relates to specific fragments of the β3 polypeptide which can be useful for example in diagnostic and ligand screening applications.

Particularly, preferred β3 fragments of interest have been selected from an analysis of the aminoacid sequence of the β3 protein. FIG. 4 comprises annotations on the β3 aminoacid sequence indicating critical regions of interest and FIG. 5 shows the three dimensional structure of β3.

β3 first forms a linear extra-cellular N-terminal domain with a single membrane spanning sequence. The 24 N-terminal amino acids of this portion of the β3 protein sequence shown in FIG. 4 as amino acids −1 to −24 include a hydrophobic region preceded by a positive residue which are typical features of a signal sequence. The location of the probable cleavage site is indicated in FIG. 4 and is supported by the presence of cysteine at position −3. The inventors believe that the β3 signal and cleavage sequences play a crucial role in the transportation of β3 and therefore may be crucial targets for the development of therapeutics and detection. These sequences therefore fall within the scope of the present invention.

Preferred peptides encoding the entire or partial β3 human or rat signal and cleavage sequence, include those of SEQ ID N° 5 and SEQ ID N° 6 which comprise the entire signal and cleavage sequence (amino acids −1 to −24 of the rat and human β3 sequences of FIG. 4). Other preferred peptides include those of SEQ ID N° 7 and SEQ ID N° 8 (aminoacids −6 to −24 of the rat and human β3 sequences of FIG. 4), those of SEQ ID N° 9 and SEQ ID N° 10 (amino acids −13 to −24 of the rat and human β3 sequences of FIG. 4), those of SEQ ID N° 11 and SEQ ID N° 12 (aminoacids −3 to −17 of the rat and human β3 sequences of FIG. 4), those of SEQ ID N° 13 and SEQ ID N° 14 (aminoacids −1 to −5 of the rat and human β3 sequences of FIG. 4) and those of SEQ ID N° 15 and SEQ ID N° 16 (aminoacids −1 to −11 of the rat and human β3 sequences of FIG. 4).

The three-dimensional structure of the central portion of the extra-cellular domain of β3, which includes aminoacids 1 to 135 of FIG. 4, is shown in FIG. 5. It was determined using as an initial model the structure of the extra-cellular domain of myelin P₀. Without wishing to be bound by any particular theory, the inventors believe that this model may provide a relatively accurate determination of the β3 structure, as the extra-cellular domain of β1, β2 and β3 shows homology to proteins which adopt a V-type Ig fold. The V-type Ig fold of myelin P₀ comprises ten β-strands (labelled A, A′, B, C, C′, C″, D, E, F and G) that form two anti-parallel sheets packed face to face. The corresponding β strands of the β3 protein are shown in FIG. 5. The inventors believe that each of these strands could have a significant impact on β3 activity or detection and hence these fall within the scope of the present invention.

The myelin P₀ model first predicts disulfide bonds at positions C21-96 and C₂₋₂₄ of β3. The former is conserved in all V-type Ig domains and is likely to be structurally important as its disruption in β1 causes an inherited epilepsy syndrome. The latter is an unusual feature in Ig domains but its probable conservation in β1 suggests functional importance (FIG. 5). It could for example help stabilise the A strand—a region implicated in α sub-unit binding. Amino acids in the A, A′ and G β3-strands form the interface with the α sub-unit. In β1, an aspartic acid residue (D5) separates strands A and A′. This aspartic acid is flanked on either side by glutamic acid residues E 4 and E 8 (FIG. 5). It has previously been shown that the simultaneous replacement of these acidic residues with neutral amino acids formed a protein that was less effective at promoting the fast gating mode of the channel. In β3, the entire A/A′ face is conserved with one exception: residue D6 of β1 is replaced with a proline in β3 (FIGS. 4 and 5). Proline tends to break beta-strands, so the gross conformation of the region should be conserved but with less pronounced negative potential. The inventors believe that this difference may suggest that β3 could favour the fast gating mode less effectively than β 1 and thereby inactivate α sub-unit opening more slowly. The inventors thus believe that aminoacids which form the β3/αsub-unit interface would play a crucial role in the modulation of sodium channels.

Interactions with other sub-units of the voltage-gated sodium channel, preferably those involved in covalent or non-covalent interactions with the α sub-unit of the voltage-gated sodium channel. Such polypeptide regions of interactions may be determined by conventional techniques well known to those skilled in the art, such as two hybrid assays as described by Fields and Song, (1989) and also in U.S. Pat. No. 5,667,973 as well as in U.S. Pat. No. 5,283,173 and in Catterall et al. (1998), the technical teachings of these publications being herein incorporated by reference. Other two-hybrid screening assays that may be performed according to the present invention are described by Young et al. (1998), the disclosure of which is also herein incorporated by reference. Other techniques useful to identify biologically relevant peptide fragments or amino acids involved in the biological activity of the β3 sub-units proteins of the invention are described by Patton et al. (1992), the disclosure of which is herein incorporated by reference.

Preferred peptides which fall within the scope of the present invention include all those which comprise the interface between αsub-unit and the rat or human β3 sub-unit. Preferred peptides encoding the p strands A, A′ and G of the β3/α sub-unit interface of the rat and human β3 protein are SEQ ID N° 22 and are SEQ ID N° 23 (amino acids −24 to 135 of the rat and human β3 sequences of FIG. 4). Other preferred peptides which encode the A and A′ D strands are those of SEQ ID N° 17 and SEQ ID N° 18 (amino acids −24 to 15 of the rat and human β3 sequences of FIG. 4), that of SEQ ID N° 19 (amino acids 2 to 10 of the human β3 sequences of FIG. 4), and those of SEQ ID No 20 and SEQ ID N° 21 (amino acids −7 to 10 of the rat and human β3 sequences of FIG. 4). Other preferred peptides encode the β strand G are SEQ ID N° 24 (amino acids 113 to 122 of the human β3 sequences of FIG. 4) and SEQ ID N° 30 (amino acids 123 to 135 of the human β3 sequences of FIG. 4).

The model also predicts that amino acids connecting β-strands B′-C, C′-C″, D-E and F-G are orientated away from the cell surface, whereas aminoacids connecting β-strands A′-B, C′-D and E-F are orientated towards the cell surface as for amino acids connecting C-C′, they are orientated approximately parallel to the cell surface. Four N-linked glycosylation sites suggest a significant potential for post-translational modification (FIG. 5).

Preferred peptides which fall within the scope of the present invention include all those polypeptides which comprise an accessible surface of the β3 sub-unit. These include polypeptides connecting β strands B′-C SEQ ID N° 25 (amino acids 24 to 36 of the human β3 sequences of FIG. 4), that of SEQ ID No 26 (amino acids 51 to 60 of the human β3 sequences of FIG. 4) connecting β strands C′-C″, that of SEQ ID N° 27 (amino acids 70 to 81 of the human β3 sequences of FIG. 4) connecting p strands D-E, that of SEQ ID N° 28 (amino acids 99 to 112 of the human β3 sequences of FIG. 4) connecting β strands F-G and that of SEQ ID N° 46 (amino acids 43 to 50) of the human β3 sequences of FIG. 4) connecting β strands C-C′.

In its C-terminal portion the β3 polypeptide comprises a hydrophobic region with strong alpha helical propensity (residues 136 to 157, see FIG. 4). This region has the properties of a transmembrane domain and is relatively well conserved in β1. The cytoplasmic region of β3 contains the sequence YLAI. The position and sequence of this motif fits the consensus for an internalization signal recognised by clathrin-coated pits. The inventors believe that the overall structural organisation of this region indicates a possible role for this class of β sub-unit in the movement of sodium channels between cellular compartments. The C-terminal position may therefore represent another target region for the development of therapeutics or detection. Preferred peptides which also fall within the scope of the present invention include all those which comprise the human and rat β3 internalisation signal. These include SEQ ID N° 31 and SEQ ID No 47 (aminoacids 158 to 191 of the rat and human β3 sequences of FIG. 4), and SEQ ID N° 32 (aminoacids 173 to 179 of the human β3 sequence of FIG. 4).

Further preferred β3 sub-unit peptide fragments are those eliciting the production of antibodies that inhibit or block the normal function of the voltage-gated sodium channel. Such inhibition or blocking of function may be measured by the method described in Example 6 and 7 of the present specification.

Other preferred peptide fragments such as defined above have at least ten contiguous amino acids of any one of the amino acid sequences of SEQ ID N°1 or SEQ ID N°2, preferably at least 12 or 15, more preferably at least 20 and most preferably at least 25 consecutive amino acids of any one of the aminoacid sequences of SEQ ID N°1 or SEQ ID N°2.

The invention also relates to a β3 sub-unit, or a peptide fragment thereof comprising aminoacid changes ranging from 1, 2, 3, 4, 5, 10, 20, 25, 30, 35, 40 substitutions, additions or deletions of one amino acid as regards to the β3 sub-unit polypeptides of anyone of the amino acid sequences of SEQ ID N°1 or SEQ ID N°2.

In the case of an amino acid substitution in the amino acid sequence of a polypeptide according to the invention, one or several consecutive or non-consecutive amino acids are replaced by “equivalent” amino acids. The expression “equivalent” amino acid is used herein to designate any amino acid that may be substituted for one of the amino acids belonging to the native protein structure without decreasing the binding properties of the corresponding peptides to the antibodies raised against the β3 sub-units polypeptides of the amino acid sequence of SEQ ID Nos 1 and 2. In other words, the “equivalent” amino acids are those which allow the generation or the synthesis of a polypeptide with a modified sequence when compared to the amino acid sequence of the native β3 sub-unit protein, said modified polypeptide being able to bind to the antibodies raised against the β3 sub-unit protein of the amino acid sequence of SEQ ID Nos 1 and 2 and/or to induce antibodies recognizing the parent polypeptide.

Alternatively, amino acid changes encompassed are those which will not abolish the biological activity of the resulting modified polypeptide. The biological activity of the modified polypeptide may be assessed, for example, as described in examples 6 and 7 of the specification.

These equivalent amino acids may be determined either by their structural homology with the initial amino acids to be replaced, by the similarity of their net charge or of their hydrophobicity, and optionally by the results of the cross-immunogenicity between the parent peptides and their modified counterparts.

The peptides containing one or several “equivalent” amino acids must retain their specificity and affinity properties to the biological targets of the parent protein, as it can be assessed by a ligand binding assay or an ELISA assay.

Examples of amino acids belonging to specific classes include Acidic (Asp, Glu), Basic (Lys, Arg, His), Non-polar (Ala, Val, Leu, Ile, Pro, Met, Phe, Trp) or uncharged Polar (Gly, Seu, Thr, lys, Tyr, Asn, Gln) amino acids.

Preferably, a substitution of an aminoacid in a β3 sub-unit polypeptide of the invention, or in a peptide fragment thereof, consists in the replacement of an aminoacid of a particular class for another aminoacid belonging to the same class.

By an equivalent amino acid according to the present invention is also contemplated the replacement of a residue in the L-form by a residue in the D form or the replacement of a Glutamic acid (E) residue by a Pyro-glutamic acid compound. The synthesis of peptides containing at least one residue in the D-form is, for example, described by Koch (1977).

A specific embodiment of a modified peptide of interest according to the present invention, includes, but is not limited to, a peptide molecule which is resistant to proteolysis. This is a peptide in which the —CONH— peptide bond is modified and replaced by a (CH₂NH) reduced bond, a (NHCO) retro inverso bond, a (CH₂—O) methylene-oxy bond, a (CH₂S) thiomethylene bond, a (CH₂CH₂) carbon bond, a (CO—CH₂) cetomethylene bond, a (CH_{O}H—CH₂) hydroxyethylene bond), a (N—N) bound, a E-alcene bond or also a —CH═CH-bond.

The invention also encompasses a β3 sub-unit polypeptide or a fragment thereof in which at least one peptide bond has been modified as described above.

The polypeptides according to the invention may also be prepared by the conventional methods of chemical synthesis, either in a homogenous solution or in solid phase. As an illustrative embodiment of such chemical polypeptide synthesis techniques, it may be cited the homogenous solution technique described by Houbenweyl (1974).

The β3 sub-unit polypeptide of interest, or a fragment thereof may thus be prepared by chemical synthesis in liquid or solid phase by successive couplings of the different amino acid residues to be incorporated (from the N-terminal end to the C-terminal end in liquid phase, or from the C-terminal end to the N-terminal end in solid phase) wherein the N-terminal ends and the reactive side chains are previously blocked by conventional groups.

For solid phase synthesis, the technique described by Merrifield (1965a; 1965b) may be used in particular.

The β3 sub-unit polypeptides of the invention and their peptide fragments of interest can be used for the preparation of antibodies.

Amplification of Nucleic Acids Encoding β3

Another object of the invention consists of a method for the amplification of a nucleic acid encoding a β3 sub-unit from a voltage-gated sodium channel, said method comprising the steps of:

(a) contacting a test sample suspected of containing the target β3 sub-unit nucleic acid, a fragment or a variant thereof, or a sequence complementary thereto, with an amplification reaction reagent comprising a pair of amplification primers located on either side of the β3 sub-unit nucleic acid region to be amplified, and

(b) optionally, detecting the amplification products.

In a first preferred embodiment of the above method, the nucleic acid encodes a human or rat β3 sub-unit, and more preferably a β3 sub-unit of any one of the amino acid sequences of SEQ ID N°1 or SEQ ID N°2.

In a second preferred embodiment of the above method, the primers comprise, or consist of, any one of the nucleotide sequences of SEQ ID N°3, SEQ ID No34 and SEQ ID N° 36 to 41.

In a third preferred embodiment of the above amplification method, the amplification product is detected by hybridization with a labelled probe having a sequence which is complementary to the amplified region.

The invention also concerns a kit for the amplification of a nucleic acid encoding a β3 sub-unit from voltage-gated sodium channel, a fragment or a variant thereof, or a complementary sequence thereto in a test sample, wherein said kit comprises:

(a) a pair of oligonucleotide primers located on either side of the β3 sub-unit nucleic acid region to be amplified;

(b) optionally, the reagents necessary for performing the amplification reaction.

In a first preferred embodiment of the kit described above, the nucleic acid encodes a human or a rat β3 sub-unit, and more preferably a β3 sub-unit of any one of the aminoacid sequences of SEQ ID N°1 and SEQ ID N°2.

In a second preferred embodiment of the above amplification kit, the amplification product is detected by hybridization with a labelled probe having a sequence which is complementary to the amplified region.

In a third embodiment of the above amplification kit, the amplification primers are selected from the nucleotide sequences of SEQ ID N°3, SEQ ID N°34 and SEQ ID N° 36 to 41.

Anti-Sense Nucleic Acids and Gene Therapy

A further object of the invention consists of antisense nucleic acids that inhibit or abolish the expression of the β3 sub-unit gene according to the invention. Preferred methods using antisense nucleic acid according to the present invention are the procedures described by Sczakiel et al. (1995).

Preferably, the antisense nucleic acids are chosen among the polynucleotides of 15-200 bp long that are complementary to the 5′end of a nucleic acid encoding a β3 sub-unit protein of the invention, preferably a human or a rat β3 sub-unit, more preferably a β3 sub-unit of any one of the aminoacid sequences of SEQ ID N°1 and SEQ ID N°2, and most preferably a nucleic acid selected from the group consisting of the nucleotides sequences of SEQ ID N°3 and SEQ ID N°4.

Preferred antisense nucleic acids according to the present invention are complementary to a sequence of the human or rat mRNAs of the β3 sub-unit that contains the translation initiation codon ATG. However, the antisense nucleic acid can also be complementary to a sequence in the 3′ or 5′ untranslated regions.

The antisense nucleic acids of the invention should have a length and a melting temperature sufficient to permit formation of an intracellular duplex having sufficient stability to inhibit the expression of the desired β3 sub-unit in the duplex. Strategies for designing antisense nucleic acids suitable for use in gene therapy are disclosed in Green et al. (1986) and Izant and Weintraub (1984), the disclosures of which are incorporated herein by reference.

Alternative suitable antisense strategies are those described by Rossi et al. (1991), in PCT Applications Nos WO 94/23 026, WO 95/04141, WO 92/18 522 and in the European Patent Application N°EP-0 572 287 A2, incorporated herein by reference.

The preferred antisense nucleic acid sequence according to the present invention is the nucleotide sequence of SEQ ID N°35.

For designing antisense nucleic acids according to the present invention, the one skilled in the art may also be guided by the teachings of the publication of Zhiqiang Zhang et al. (1998), the disclosure of which is herein incorporated by reference.

Without wishing to be bound by any particular theory, the inventors believe that one way in which modification of sodium channels including a β3 sub-unit can alter excitability would be the following mechanism. The decay of the sodium current can be fitted by at least two exponents. The major component of these two currents is significantly shortened by co-expression of the β3 sub-units. This is due to the β3 sub-unit significantly shifting the voltage activation curve in a positive direction allowing more rapid repolarization of the membrane potential. Thus an increase in the number of events or transfer of information down the axon occurs. The high density of sodium channels expressed in neurones allows conduction of action potentials to occur over the entire cell surface. Transient removal of sodium channels from the membrane may afford a reversible method of changing the gain function of a nerve terminal to depolarising input.

Consequently, the inventors believe that inhibition of the expression of the β3 sub-unit of the invention, for example via an antisense strategy, may affect the expression and/or the surface expression of the voltage-gated sodium channel of which the β3 sub-unit is part, and consequently affect the expression and the biological activity of the whole voltage-gated sodium channel. Such a voltage-gated sodium channel inhibition may be useful for preventing or curing diseases like epilepsy, hyperalgesia and cardiovascular diseases.

Additionally, increasing the inactivation of sodium channels will lead to a damping effect on neuronal excitability.

Moreover, it is suggested that the β3 sub-units are tightly bound to the α pore and that trafficking of the complex to the appropriate place requires the β3 sub-unit. Thus, a modification, preferably an inhibition, of the β3-α complex may be used to increase sequestration to an intracellular site or reduced trafficking of a and a to terminal membrane reagents. This would reduce excitability since current density during depolarisation would be insufficient to maintain propagation of the action potential.

Furthermore, changing the expression of the β3 sub-unit would cause a disruption of sodium channel function in an injured region.

Another object of the invention is the use of the nucleic acids encoding the β3 sub-unit or a biologically active peptide fragment thereof in gene therapy, by insertion of the fully functional gene by a vector delivery system that would result in the repair of a damaged area. In order to affect expression of the nucleic acids encoding a β3 sub-unit protein of the invention, these nucleic acids must be delivered into a cell. This delivery may be accomplished in vitro, as in laboratory procedures for transforming cell lines, or in vivo or ex vivo, as in the treatment of certain disease states, particularly disease states related to a dysfunction in the voltage-gated sodium channels, and more particularly disease states such as pain, epilepsy, stroke, ischemia, hyperalgesia, cardiovascular disease and Jacobsen Syndrome, Familial Nonchromaffin Paraganglioma, Phenylketonuria due to PTS deficiency and Charcot Marie Tooth disease.

One mechanism is viral infection where the nucleic acid to be expressed is encapsulated in an infectious viral particle. Several non-viral methods for the transfer of polynucleotides into cultured mammalian cells are also contemplated by the present invention, and include, without being limited to, calcium phosphate precipitation: Graham et al., (1973); Chen et al., (1987), DEAE-dextran Gopal: (1985), electroporation: Tur-Kaspa et al., (1986); Potter et al., (1984), direct micro-injection: Harland et al., (1985), and DNA-loaded liposomes: Nicolau et al., (1982); Fraley et al., (1979).

Once the nucleic acid to be expressed has been delivered into the cell, it may be stably integrated into the genome of the recipient cell. This integration may be in the right location and orientation via homologous recombination (gene replacement) or it may be in a random, non specific location. In yet further embodiments, the nucleic acid may be stably maintained in the cell as a separate, episomal segment of DNA. Such nucleic acid segments or episomes encode sequences sufficient to permit maintenance and replication independent of, or in synchronization with the host cell cycle. A suitable gene targeting technique is described in Russel (1998), the disclosure of which is herein incorporated by reference.

One specific embodiment of a method for delivering a nucleic acid to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect.

Compositions for use in vitro and in vivo comprising a naked polynucleotide are described in PCT Application N° WO 90/11 092 (Vical Inc.) as well as in the articles of Tacson et al. (1996) and of Huygen et al., (1996), the disclosures of which are herein incorporated by reference.

Another object of the invention consists of a composition for the in vivo production of a β3 sub-unit protein or a biologically active peptide fragment thereof. Such a composition may comprise a naked polynucleotide operatively coding for this polypeptide, in solution in a physiologically acceptable carrier, and suitable for introduction into a tissue to cause cells of the tissue to express a functional β3 sub-unit protein or a peptide fragment thereof and thus a functional voltage-gated sodium channel.

The amount of vector to be injected to the desired host organism varies according to the site of injection. As an indicative dose, it will be injected between 0.1 and 100 μg of the vector in an animal body, preferably a mammal body, and preferably a human body.

In another embodiment of a gene therapy method of the invention, the nucleic acid that operatively expresses the β3 sub-unit protein or a biologically active peptide fragment thereof may be introduced in vitro in a host cell, preferably in a host cell previously harvested from the animal to be treated and more preferably a somatic cell such as a muscle cell or a neuronal cell. In a subsequent step, the cells that have been transformed with the nucleic acid encoding the β3 sub-unit protein or its peptide fragment of interest is reintroduced into the animal body in order to deliver the recombinant protein within the body either locally or systemically.

Therefore, the invention is also directed to a composition containing a nucleic acid selected from the group of nucleic acids described therein, in combination with one or several physiologically acceptable carriers, such as those well known from the one skilled in the art.

Recombinant Expression Vectors

The present invention also encompasses a family of recombinant vectors comprising any one of the nucleic acids described herein. Thus, the invention further deals with a recombinant vector comprising a nucleic acid selected from the group consisting of:

(a) a purified or isolated nucleic acid encoding a β3 sub-unit from a voltage-gated sodium channel, preferably a human or a rat β3 sub-unit, and more preferably a polypeptide having at least 80% amino acid identity with a polypeptide selected from the group consisting of the amino acid sequences of SEQ ID N°1 and SEQ ID No2, or a sequence complementary thereto;

(b) a purified or isolated nucleic acid having at least 90% nucleotide identity with a polynucleotide selected from the group consisting of the nucleotide sequences of SEQ ID N°3 and SEQ ID N°4, or a sequence complementary thereto;

(c) a purified or isolated polynucleotide comprising at least 10 consecutive nucleotides of a nucleic acid described in (a) or (b), or a sequence complementary thereto; and

(d) a purified or isolated polynucleotide selected from the group consisting of polynucleotides encoding one of the peptide fragments of β3 corresponding to SEQ ID No 5 to 32 and SEQ ID N°46 and 47.

In a first preferred embodiment a recombinant vector of the invention is used to amplify the inserted polynucleotide derived from the nucleic acid encoding a β3 sub-unit of the invention in a suitable host cell, this polynucleotide being amplified every time the recombinant vector replicates.

A second preferred embodiment of the recombinant vectors according to the invention consists of expression vectors comprising a nucleic acid encoding a β3 sub-unit of the invention, preferably a nucleic acid encoding a human or a rat β3 sub-unit, and more preferably a nucleic acid encoding a polypeptide selected from the group consisting of the amino acid sequences of SEQ ID N°1 and SEQ ID N°2, and most preferably a nucleic acid selected from the group consisting of the nucleotide sequences of SEQ ID N°3 and SEQ ID N°4.

Recombinant expression vectors comprising a nucleic acid encoding the peptide fragments of a β3 sub-unit that are specified in the present specification are also part of the invention.

Within certain embodiments, expression vectors can be employed to express the β3 sub-unit of the invention or a peptide fragment thereof which can then be purified and for example, be used as a immunogen in order to raise specific antibodies directed against said β3 sub-unit protein or a peptide fragment thereof.

In another embodiment, the expression vectors are used for constructing transgenic animals and also for gene therapy, notably for antisense therapy.

Expression requires that appropriate signals are provided in the vectors, said signals including various regulatory elements such as enhancers/promoters from both viral and mammalian sources that drive expression of the genes of interest in host cells. The regulatory sequences of the expression vectors of the invention are operably linked to the nucleic acid encoding the β3 sub-unit protein of interest or a peptide fragment thereof.

As used herein, the term “operably linked” refers to a linkage of polynucleotide elements in a functional relationship. For instance, a promoter or an enhancer is operably linked to a coding sequence if it affects the transcription of the coding sequence.

More precisely, two DNA molecules (such as a polynucleotide containing a promoter region and a polynucleotide encoding a desired polypeptide or polynucleotide) are said to be “operably linked” if the nature of the linkage between the two polynucleotides does not: (1) result in the introduction of a frame-shift mutation or (2) interfere with the ability of the polynucleotide containing the promoter to direct the transcription of the coding polynucleotide.

Generally, recombinant expression vectors will include origins of replication, selectable markers, permitting transformation of the host cell, and a promoter derived from a highly expressed gene to direct transcription of a downstream structural sequence. The heterologous structural sequence is assembled in an appropriate frame with the translation, initiation and termination sequences, and preferably a leader sequence capable of directing sequences of the translated protein into the periplasmic space or the extra-cellular medium.

In a specific embodiment wherein the vector is adapted for transfecting and expressing desired sequences in mammalian host cells, preferred vectors will comprise an origin of replication from the desired host, a suitable promoter and an enhancer, and also any necessary ribosome binding sites, polyadenylation site, transcriptional termination sequences, and optionally 5′-flanking non-transcribed sequences.

DNA sequences derived from the SV 40 viral genome, for example SV 40 origin, early promoter, enhancer, and polyadenylation sites may be used to provide the required non-transcribed genetic elements.

In another embodiment of a recombinant expression vector of the invention, the 5′-flanking non transcribed sequence may comprise a polynucleotide selected from the group consisting of:

(1) the nucleic acid beginning at the nucleotide in position 1 and ending at the nucleotide in position 362 of the nucleotide sequence of SEQ ID N°3;

(2) the nucleic acid beginning at the nucleotide in position 1 and ending at the nucleotide in position 375 of the nucleotide sequence of SEQ ID N°4.

Additionally, a recombinant expression vector of the invention advantageously also comprises an untranscribed polynucleotide located at the 3′end of the coding sequence (ORF), this 3′-UTR polynucleotide being useful for stabilizing the corresponding mRNA or for increasing the expression rate of the vector insert if this 3′-UTR harbors regulation signal elements such as enhancer sequences.

A preferred 3′-UTR sequence will be selected from the group consisting of the 3′-UTR sequences contained in the nucleotide sequences of SEQ ID N°3 and SEQ ID N°4.

Thus, a further object of the invention consists of a 3′-UTR nucleic acid selected from the group consisting of:

(1) the nucleic acid beginning at the nucleotide in position 1011 and ending at the nucleotide in position 2220 of the nucleotide sequence of SEQ ID N°3;

(2) the nucleic acid beginning at the nucleotide in position 1024 and ending at the nucleotide in position 1261 of the nucleotide sequence of SEQ ID N°4.

Suitable promoter regions used in the expression vectors according to the invention are chosen taking into account the host cell in which the heterologous nucleic acids have to be expressed.

A suitable promoter may be heterologous with respect to the nucleic acid for which it controls the expression, or alternatively can be endogenous to the native polynucleotide containing the coding sequence to be expressed.

Additionally, the promoter is generally heterologous with respect to the recombinant vector sequences within which the construct promoter/coding sequence has been inserted.

Preferred bacterial promoters are the LacI, LacZ, T3 or T7 bacteriophage RNA polymerase promoters, the lambda PR, PL and trp promoters (a EP-0 036 776), the polyhedrin promoter, or the p 10 protein promoter from baculovirus (kit Novagen; Smith et al., (1983); O'Reilly et al. (1992).

Preferred selectable marker genes contained in the expression recombinant vectors of the invention for selection of transformed host cells are preferably dehydrofolate reductase or neomycin resistance for eukaryotic cell culture, TRP1 for S. cerevisiae or tetracycline, rifampicin or ampicillin resistance in E. coli, or Levamsaccharase for Mycobacteria, this latter marker being a negative selection marker.

Preferred bacterial vectors of the invention are listed hereafter as illustrative but not limitative examples:

pQE70, pQE60, pQE-9 (Quiagen), pD10, phagescript, psiX174, p.Bluescript SK, pNH8A, pNH16A, pNH18A, pNH46A (Stratagene); pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIA express).

Preferred bacteriophage recombinant vectors of the invention are P1 bacteriophage vectors such as described by Sternberg N. L. (1992; 1994).

A suitable vector for the expression of a β3 sub-unit polypeptide of the invention or a fragment thereof, is a baculovirus vector that can be propagated in insect cells and in insect cell-lines. A specific suitable host vector system is the pVL 1392/1393 baculovirus transfer vector (Pharmingen) that is used to transfect the SF9 cell line (ATCC N°CRL 1711) which is derived from spodoptera frugiperda.

The recombinant expression vectors from the invention may also be derived from an adenovirus such as those described by Feldman and Steig. (1996) or Ohno et al. (1994).

Another preferred recombinant adenovirus according to this specific embodiment of the present invention is the human adenovirus type two or five (Ad 2 or Ad 5) or an adenovirus of animal origin (French Patent Application n°FR 93 05 954).

Particularly preferred retrovirus as for the preparation or construction of retroviral in vitro or in vivo gene delivery vehicles of the present invention include retroviruses selected from the group consisting of Mink-Cell Focus Inducing Virus, murine sarcoma virus, and Ross Sarcoma Virus. Other preferred retroviral vectors are those described in Roth et al. (1996), in PCT Application WO 93/25 234, in PCT Application WO 94/06920, and also in Roux et al. (1989), Julan et al. (1992) and Nada et al. (1991).

Yet, another viral vector system that is contemplated by the invention consist in the adeno associated viruses (AAV) such as those described by Flotte et al. (1992), Samulski et al. (1989) and McLaughlin et al. (1996).

Thus, a further object of the invention consists of a recombinant expression vector comprising a nucleic acid encoding a β3 sub-unit from a voltage-gated sodium channel or a peptide fragment thereof or a variant thereof, wherein said nucleic acid is operably linked to a promoter sequence.

In a preferred embodiment, this nucleic acid encodes a rat or a human β3 sub-unit, and preferably a β3 sub-unit of any one of the aminoacid sequences of SEQ ID No1 and SEQ ID N°2, or a variant or a peptide fragment thereof. Preferred fragments include those of SEQ ID N° 5 to 32. and SEQ ID N° 46 and 47. In a most preferred embodiment, this nucleic acid comprises any one of the nucleotide sequences of SEQ ID N° 3 and SEQ ID N° 4.

Host Cells Expressing β3

Host cells that have been transformed or transfected with one of the nucleic acids described herein, or with one of the recombinant vector, particularly recombinant expression vector, described herein are also part of the present invention.

Are included host cells that are transformed (prokaryotic cells) or are transfected (eukaryotic cells) with a recombinant vector such as one of those described above. Preferred host cells used as recipients for the expression vectors of the invention are the following:

(a) prokaryotic host cells: Escherichia coli, strains. (i.e. DH5-α, strain) Bacillus subtilis, Salmonella typhimurium and strains from species like Pseudomonas, Streptomyces and Staphylococcus;

(b) eukaryotic host cells: HeLa cells (ATCC N°CCL2; N°CCL2.1; N°CCL2.2), Cv 1 cells (ATCC N°CCL70), COS cells (ATCC N°CRL 1650; N°CRL 1651), Sf-9 cells (ATCC N°CRL 1711), C127 cells (ATCC N°CRL-1804), 3T3 cells (ATCC N°CRL-6361), CHO cells (ATCC N°CCL-61), human kidney 293 cells (ATCC N° 45504; N°CRL-1573), BHYK (ECACC N°84100 501; N°84111301), PC12 (ATCC N° CRL-1721), NT2, SHSY5Y (ATCC N° CRL-2266), NG108 (ECACC N°88112302) and F11, SK-N-SH (ATCC N° CRL-HTB-11), SK-N-BE(2) (ATCC N° CRL-2271), IMR-32 (ATCC N° CCL-127). A preferred system to which the gene of the invention can be expressed are cell lines such as COS cells, 3T3 cells, HeLa cells, 292 cells and CHO cells. A preferred system for the efficient expression of β3 involves the use of CHO cell lines. The gene can be expressed through an endogenous promoter of native CHO, or through an exogenous promoter. Suitable exogenous promoters include such as SV40 and CMV, or perhaps a eucaryotic promoter such as the tetracycline promoter. The preferred promoter being CMV.

In a specific embodiment of the host cells described above, these host cells have also been transfected or transformed with a polynucleotide or a recombinant vector allowing the expression of another voltage-gated sodium channel sub-unit, preferably a sub-unit of the alpha type, and more preferably a sub-unit of the α2 type, such as described in Example 4. Suitable co-expression procedures are also described in Makielski et al. (1996), and by Qu et al. (1995), the disclosure of which is herein incorporated by reference.

The present invention also concerns a method for producing one of the β3 sub-unit polypeptides or peptides described herein and especially a polypeptide selected from the group consisting the aminoacid sequences of SEQ ID N°1 or SEQ ID N°2, wherein said method comprises the steps of:

(a) inserting the nucleic acid encoding the desired β3 sub-unit polypeptide or peptide fragment thereof in an appropriate vector;

(b) culturing, in an appropriate culture medium, a host cell previously transformed or transfected with the recombinant vector of step (a);

(c) harvesting the culture medium thus conditioned or lyse the host cell, for example by sonication or by an osmotic shock;

(d) separating or purifying, from said culture medium, or from the pellet of the resultant host cell lysate, the thus produced β3 sub-unit polypeptide of interest.

In a first preferred embodiment of the above method, the nucleic acid to be inserted in the appropriate vector has previously undergone an amplification reaction, using a pair of primers.

Preferred primers used for such an amplification reaction are the primers of the nucleotide sequences of SEQ ID N°3 and SEQ ID N°34.

In a second preferred embodiment of the above method, the polypeptide thus produced is further characterized, for example by binding onto an immuno-affinity chromatography column on which polyclonal or monoclonal antibodies directed to the β3 sub-unit polypeptide or a peptide fragment thereof have previously been immobilised.

Purification of the recombinant β3 sub-unit proteins according to the present invention or a peptide fragment thereof may be carried out by passage onto a nickel or copper affinity chromatography column.

In another embodiment, the β3 sub-unit polypeptides or peptide fragments thus obtained may be purified, for example, by high performance liquid chromatography, such as reverse phase and/or cationic exchange HPLC, as described by Rougeot et al. (1994).

The reason to prefer this kind of peptide or protein purification is the lack of by-products formed in the elution samples which renders the resultant purified protein or peptide more suitable for therapeutic use.

Antibodies to β3

Polyclonal antibodies may be prepared by immunization of a mammal, especially a mouse or a rabbit, with a polypeptide or peptide according to the invention that is combined with an adjuvant of immunity, and then by purifying the specific antibodies contained in the serum of the immunized animal on an affinity chromatography column on which has previously been immobilized the polypeptide that has been used as the antigen.

Monoclonal antibodies may be prepared from hybridomas according to the technique described by Kohler and Milstein (1975).

The present invention also deals with antibodies produced by the trioma technique and by the human B-cell hybridoma technique, such as described by Kozbor et al. (1983).

Antibodies of the invention also include chimeric single chain Fv antibody fragments (U.S. Pat. No. 4,946,778; Martineau et al., (1998), antibody fragments obtained through phage display libraries Ridder et al. (1995) and humanized antibodies (Leger et al., (1997)).

Antibody preparations obtained according to either protocols are useful in quantitative immuno assays for determining the presence of antigen bearing substances in biological samples. The antibodies may also be used in therapeutic compositions aimed to inhibit the biological activity of a β3 sub-unit from a voltage-gated sodium channel.

Consequently, the invention is also directed to a method for specifically detecting the presence of a β3 sub-unit from a voltage-gated channel in a sample, said method comprising the following steps of:

(a) bringing into contact a sample to be assayed with an antibody directed against a β3 sub-unit protein or to a peptide fragment thereof;

(b) detecting the antigen-antibody complex formed.

The invention also concerns a kit for detecting in vitro the presence of a β3 sub-unit polypeptide or a fragment thereof in a sample, wherein said kit comprises an antibody directed against a β3 sub-unit polypeptide or a peptide fragment thereof.

In a preferred embodiment, the kit further comprises a reagent allowing the detection of the antigen-antibody complexes formed, said reagent carrying optionally a label, or being able to be recognized itself by a labelled reagent, particularly in the case when the above mentioned antibody is not itself labelled.

The antibodies of the present invention are also useful as therapeutic agents capable of blocking the biological activity of brain voltage-gated sodium channel.

Thus, another object of the invention consists of a composition containing an antibody as defined herein, in combination with one or several physiologically acceptable carriers, such as those well known from the one skilled in the art.

Screening for β3 Ligands

The present invention also concerns methods for screening ligand substances or molecules that are able to modulate the biological activity of a voltage-gated sodium channel containing a β3 sub-unit of the invention.

Production of β3 or a Fragment Thereof.

The β3 protein or fragments thereof can be prepared using recombinant technology, cell lines or chemical synthesis. Recombinant technology and chemical synthesis of the β3 sub-unit or fragments thereof can allow the modification of the gene encoding the β3 sub-unit to include such features as recognition tags, cleavage sites and modifications of the β3 sub-unit or fragments thereof. For efficient polypeptide production, the recombinant expression system should allow the β3 polypeptide to be expressed and transported at the cell surface in a functional form or allow production of β3 sub-unit fragments which can be purified. Preferred cell lines are those which allow high levels of expression of β3 sub-unit or fragments thereof. Such cell lines include common mammalian cell lines such as Cho cells and Cos cells, etc or more specific neuronal cell lines such as PC12. However, other cell types which are commonly used for recombinant protein production such as insect cells, amphibian cells such as oocytes, yeast and procaryotic cell lines such as E.coli can also be considered.

The β3 sub-unit or fragments thereof can be utilised in a ligand screen either as a purified protein, as a protein chimera such as those described in phage display, as a cell membrane (lipid or detergent) preparation, or in intact cells.

The β3 sub-unit or fragment thereof can be utilised in a functional screen format or ligand binding screen format. Examples of both screening formats are provided below.

Functional Screening Methods

A first embodiment of a functional screen comprises of the following steps:

(a) obtaining a recombinant host cell co-expressing a β3 sub-unit or a fragment thereof and a functional a sub-unit, preferably an α2 sub-unit of a voltage-gated sodium channel, or a fragment thereof;

(b) bringing into contact said recombinant host cell with a substance or molecule to be tested; and

(c) measuring an electrical parameter within the recombinant host cell brought into contact with the substance or molecule to be tested through a voltage clamp technique or measurement of membrane potential by voltage sensitive fluorescent dyes.

A first preferred electrical parameter to be measured is the inactivation potential.

A second preferred electrical parameter to be measured is the inactivation time.

A third preferred electrical parameter to be measured is the rate of recovery of the sodium channel.

Measurement of membrane potential can be carried out using one of the techniques described in the following references, which describe the utility of voltage sensitive dyes: Biophys-J. 1989 December; 56(6): 1053-69, Biochemistry 1989 May 30; 28(11): 4536-9, Chem-Biol. 1997 April; 4(4): 269-77, Biophys-J. 1995 October; 69(4): 1272-80. All these publications are incorporated herein by reference.

Another embodiment of a functional screening method comprises the following steps:

(a) obtaining a recombinant host cell co-expressing a β3 sub-unit or a fragment thereof and a functional α sub-unit, preferably an α2 sub-unit of a voltage-gated sodium channel, or a fragment thereof;

(b) bringing into contact said recombinant host cell with a substance or molecule to be tested; and

(c) measuring the change in sodium flux within the recombinant host cell brought into contact with the substance or molecule to be tested through a sodium flux measuring technique by sodium sensitive dyes such as SBFI.

A first preferred parameter to be measured to be measured is the increase in sodium concentration within the cell.

A second preferred parameter to be measured is the decrease in sodium concentration within the cell.

A third preferred parameter to be measured is the rate of recovery of the sodium channel.

Measurement of changes in intracellular sodium concentration can be carried out using one of the techniques described in the following references, which describe the utility sodium sensitive dyes: Witkowski et al Nature 1998, 392, 78, Itoh et al, J. Neuroscience 1998, 71, 112, Mittmann et al, J. Neurophysiol 1997, 78, 1188 and David et al J. Physiol. 1997, 295. All these publications are incorporated herein by reference.

A further embodiment of a functional screening method comprises the following steps:

(a) obtaining a recombinant host cell expressing a functional α sub-unit, preferably an α2 sub-unit of a voltage-gated sodium channel, or a fragment thereof,

(b) bringing into contact said recombinant host cell with a substance or molecule to be tested and a fragment of the β3 sub-unit, preferably a fragment of the β3 sub-unit from which at least the transmembrane domain has been removed; and

(c) measuring an electrical parameter within the recombinant host cell brought into contact with the substance or molecule to be tested through a voltage clamp technique or measurement of membrane potential by voltage sensitive fluorescent dyes in a similar manner to that described above.

Another embodiment of a functional screening method comprises the following steps:

(a) obtaining a recombinant host cell expressing a functional a sub-unit, preferably an α2 sub-unit of a voltage-gated sodium channel, or a fragment thereof;

(b) bringing into contact said recombinant host cell with a substance or molecule to be tested and a fragment of the β3 sub-unit, preferably a fragment of the β3 sub-unit from which at least the transmembrane domain has been removed; and

(c) measuring the change in sodium flux within the recombinant host cell brought into contact with the substance or molecule to be tested through through a sodium flux measuring technique by sodium sensitive dyes such as SBFI in a similar manner to that described above.

Ligand Binding Screening Method

A typical embodiment of a ligand binding screen comprises of the following steps;

(a) contacting the ligand with the β3 sub-unit or a fragment thereof.

The β3 polypeptide can be part of an intact cell, membrane preparation or purified polypeptide. The ligand can be a peptide/protein/antibody or chemical entity. The principal property the ligand must have is that it must recognise and bind to a binding site determined by the β3 aminoacid sequence.

Optionally, excess non β3 bound ligand can be removed by separation. Separation can take the form of washing/filtering or centrifugation (to pellet the β3 protein). In this latter case, the supernatant can then be removed and the β3 re-suspended in buffer.

(b) contact the medium containing the ligand and the β3 protein or a fragment thereof with a β3 substrate and allow binding to occur.

A property of the substrate must be that it is detectable and quantifiable. To achieve this the substrate can be a chromophore or radio, fluorescent, phosphorescent, enzymatically or antibody labelled. If the substrate is not directly detectable it must be amenable to detection and quantification by secondary detection, which may employ the above technologies.

Optionally, unbound substrate can be removed from the mixture as described above.

(c) measurement of substrate binding

Binding of the ligand modifies the interaction of the substrate with the β3 binding site and decreases affinity of substrate for the binding site. The difference between the observed amount of substrate bound relative to the theoretical maximum amount of substrate bound is a reflection of the amount and affinity of ligand bound to the substrate-binding site. The mechanism of detection of substrate is determined by it properties.

Alternatively, the amount of ligand bound to the β3 sub-unit or a fragment thereof can be determined by a combination of chromatography and mass spectroscopy.

The amount of ligand bound to the β3 sub-unit or a fragment thereof can also be determined by direct measurement of the change in mass upon ligand or substrate binding to β3. This could be achieved with technologies such as Biocore (Amersham Pharmacia).

Alternatively, the β3 sub-unit or a fragment thereof, the substrate or the ligand can be fluorescently labeled and association of β3 with the ligand can be followed by changes in Fluorescence Energy Transfer (FRET).

In a first preferred embodiment of the above methods, substances or molecules of interest are selected among those which induce changes in the activation potential, the inactivation time, or the rate of recovery of the sodium channel.

Preferred molecules or substances are those inducing a decrease in the inactivation potential, and/or a decrease in the rate of inactivation, and/or which decreases the rate of recovery from inactivation, as compared with the same measures performed in the absence of the substance of molecule to be tested.

Molecules that may be assayed according to the method described above comprise, but are not limited to, voltage-dependent channel blockers, tetrodotoxin, lidocaine, phenyloin, carbamazepine, lamotrigine, zonisamide, riluzole, lifarizine, ralitoline, flunarizine, verapamil and carvedilol.

Other substances that may be assayed according to the method described above are molecules from the phenylacetamide familly, 6-Iodoamiloride.

Sodium channel openers may also represent good candidate molecules, such as for example carsatrin or BDF-9148 (Beiersdorf).

Therapeutic molecules active on neuropathic pain or migraine may also be used, such as CNS-5161 (Cambridge Neuroscience's).

The invention also concerns a kit for screening substances or molecules capable of modulating the biological activity of voltage-gated sodium channel containing a β3 sub-unit.

In a first embodiment, the kit comprises a recombinant host cell co-expressing a β3 sub-unit or a fragment thereof and an α sub-unit, preferably an α2 sub-unit, or a fragment thereof.

In a second embodiment, the kit comprises a recombinant host cell expressing a functional α sub-unit, preferably an α2 sub-unit, or a fragment thereof, and a fragment of the β3 sub-unit, preferably a fragment from which at least the transmembrane domain has been removed.

In a third embodiment, the kit comprises the β3 sub-unit or a fragment thereof and a suitable β3 substrate.

### EXAMPLES

### Example 1

### Isolation and Cloning of the cDNA Encoding the Sodium Channel β3 Sub-Unit from Rat

A variant of the rat pheochromocytoma cell line PC12 that has lost many of its neuroendocrine properties has been studied. Subtractive cloning to isolate cDNAs corresponding to mRNA expressed in normal PC12 cells but missing from the variant were isolated whilst identifying novel neuroendocrine-specific transcripts.

Total RNA was prepared from wild-type PC12 and variant cell lines as described by Chomcznski and Saatchi (Chomczynski and Saachi, 1987). Poly A(+) RNA was purified from the total RNA by Oligo dT Cellulose column chromatography (Pharmacia UK) (Aviv and Lader 1972). The yield of mRNA from each cell line was calculated spectrophotometrically before proceeding with subtractive hybridisation using the technique of PCR select (Clonetech, USA; Diatchenko et al. 1996). Amplified cDNA fragments derived from genes differentially expressed in the wild-type cells were subcloned into the pTAdv plasmid (Clonetech) (Mead et al. 1994) and transformed into E. coli strain XLI blue (Bullock et al. 1987) to create a cDNA fragment library. Plasmid minipreps from randomly picked subclones were subjected to automated DNA sequencing and screened through DNA data base searches.

Full length coding sequence of rat β03 was isolated by screening a rat brain cDNA library with a partial clone isolated by PCR select. The rat brain cDNA library in lambdaZap (Short et al. 1988) was plated on E. coli strain c600hfl (Huynh et al. 1985) and phage plaques were screened with a 400 bp ³²P-labelled XbaI-SacI. cDNA fragment derived from the PCR select clone. Out of approximately 250,000 plaques, a single positive phage clone was isolated by plasmid rescue in pBluescript km288 plasmid using the E.coli XPORT, XLOLR system. (Alting-Mees & Short 1994). Both strands of the cDNA insert in km288 were subjected to automated DNA sequencing on both strands using M13 primers and internal sequence-specific primers. The resulting sense nucleic acid sequence is herein referred to as SEQ ID N°3.

### Example 2

### Isolation and Cloning of the cDNA Encoding the Sodium Channel β3 Sub-Unit from Human

The human homologue of the novel rat β3 sub-unit was cloned from a human striatal Lambda ZAP II cDNA library obtained from Stratagene. The entire nucleotide sequence encoding the rat β3 open reading frame was amplified by PCR. This was performed using 20 mer oligonucleotides:

| | |
|---|---|
| 5′-ATGCCTGCCTTCAACAGATTGC-3′ | SEQ ID N°33 |
| (362-383 bp of the rat β3 sequence) | |
| as the forward primer, and | |
| 5′-TTATTCCTCCACAGGTACCA-3′ | SEQ ID N°34 |
| (1007-1026 bp of the rat β3 sequence) | |
| as the reverse primer. | |

The double stranded PCR product produced was radiolabelled by nick translation with [α³²P] dATP and [α³²P] d CTP and used to probe 10⁶ primary plaques bound to nitrocellulose filters in a standard hybridization buffer containing 25% formamide. Single plaques giving rise to positive hybridizations were isolated and insert cDNA sequenced on an ABI 310 DNA analyser. The resulting sense nucleic acid sequence is referred to as SEQ ID N°4.

### Example 3

### Tissue Expression by Polymerase Chain Reaction

Total RNA was prepared from adult rat tissues and PC12 cells, treated with DNAase I to remove genomic contamination, and reverse transcribed using MMLV reverse transcriptase with anchored oligo dT primer according to the manufacturer's recommendations. Approximately 0.5 and 5 ng of cDNA was separately subjected to PCR amplification using primers specific for rat β1 (accession number m91808), rat β3 (accession number AJ243395) and, to ensure similar amounts of cDNA were used in each reaction, rat α-tubulin (accession number V01226). The primers used were chosen to correspond to unique sequences in the 3′ untranslated region of each β subunit:

β1 forward primer (nucleotides 1103-1120) SEQ ID N°36

5′ GGTGAAGCAATATGGCCG 3′,

reverse primer (nucleotides 1317-1300) SEQ ID N°37

5′ AGATGAGGCCCAGAACCC 3′,

β3 forward primer (nucleotides 1942-1961) SEQ ID N°38

5′ GGAAGCTGACTGCCACAGAT 3′,

reverse primer (nucleotides 2209-2190) SEQ ID N°39

5′ CCTGGGGGACTTTACAAACA 3′,

α-tubulin forward primer (nucleotides 298-316) SEQ ID N°40

5′ CACTGGTACGTGGGTGAGG 3′,

reverse primer (nucleotides 469-448) SEQ ID N°41

5′ TTTGACATGATACAGGGACTGC 3′. PCR was performed as described above except that amplification reactions included 0.125 μl Taqstart antibody (CLONETECH). A control amplification lacking cDNA was also included. After amplification the products were separated on 2.5% agarose gels and visualised using ethidium bromide (FIG. 2).

### Example 4

### In Situ Hybridization Studies of Distribution of β3 Sub-Unit

Whole brains were dissected from adult (150- to 200-g) Wistar rats and snap frozen on dry ice. 10 μm cryostat sections were thaw mounted onto poly-1-lysine coated slides, fixed with 4% paraformaldehyde in PBS (pH 7.4), dehydrated and stored under ethanol until hybridization. The sequence and location of the anti-sense oligonucleotides used for analysis were as follows:

| rat β1 (nucleotides 1296-1252) | |
|---|---|
| 5′GCTTGATGGGGTGAAGAGGGGTCGGGACAGGGACAGTAGTGGGC | SEQ ID N° 42 |
| 3′, | |
| rat β 3 (nucleotides 389-345) | |
| 5′GGGGAAGCAATCTGTTGAAGGCAGGCATCTTTTCCACCGTAAGCG | SEQ ID N° 35 |
| 3′, | |
| rat α IIA (nucleotides 1659-1615) | |
| 5′GCAGAATCCAGAGACTTCAGCGGGGCAGGCGGGATAGGTGTTTTC | SEQ ID N° 43 |
| 3′. | |

Oligonucleotides were 3′ end-labelled with [³⁵S]dATP (Amersham Pharmacia; 1000 Ci/mmol) by terminal deoxynucleotidyl transferase (Roche Molecular Biochemicals; ref. Ausubel, F. R et al 1989) and used for hybridization at a concentration of 400,000 cpm/100 μl of hybridization buffer. To confirm the specificity of the hybridizations, 100 fold excess of unlabelled oligonucleotide was added to the hybridization buffer in addition to the radiolabelled probe. Slides were air dried and hybridized overnight at 42° C. in 150 μl buffer containing 50% formamide, 10% dextran sulfate, 50 mM DTT, 1× Denhardt's solution, 0.5 mg/ml denatured salmon sperm DNA and 0.5 mg/ml polyadenylic acid (all Sigma, Poole, U.K.). Sections were washed in 1×SSC at 55° C. for 30 minutes, rinsed in 1×SSC, 0.1×SSC dehydrated and apposed to Kodak Biomax™ MR X-ray film (Amersham Pharmacia) for 10 days.

Sections were observed and photographed using a Polyvar microscope (Reichert-Jung) with camera attached FIG. 3.

The sequence depicted in SEQ ID N°35 is the antisense radiolabelled oligonucleotide probe used in the in situ hybridization experiments, unique to target sequence as confirmed by FASTA search (NCBI). This allows the detailed distribution as shown in table 1 to be determined and changes in distribution to be detected. mRNA on adult rat brain sections (emulsion dipped sections and autoradiographs), were rated for relative optical density (ROD): ++++++, +++++, very abundant; ++++, abundant; +++, moderate; ++,+, low; ½, just above background; 0, not detectable. n=3-6. VBD—Nucleus of the Vertical limb of the Diagonal Band, VTA—Ventral Tegmental Area, APTD—Anterior Pretectal Nucleus (dorsal part), DpMe—Deep Mesencaphalic Nucleus.

TABLE 1

| | αIIA | β1 | β3 | | αIIA | β1 | β3 |
|---|---|---|---|---|---|---|---|
| OLFACTORY SYSTEM | | | | THALAMUS | | | |
| Anterior Olfactory | +++ | ½ | +++ | Reticular Nucleus | 0 | ½ | 0 |
| Olfactory Tubercle | +++ | ½ | +++ | Medial GeniculateNuclei | ½ | ++ | 0 |
| Piriform Cortex | ++++++ | ++ | ++++ | Ventrolateral Geniculate | ++ | + | ½ |
| NEOCORTEX | | | | Dorsal Lateral Nuclei | 0 | + | 0 |
| Layer 2 | +++ | + | +++ | Anteroventral/dorsal Nuclei | ++ | ++ | 0 |
| Layer 3 | +++ | + | +++ | Ventral Nuclei | + | ½ | 0 |
| Layer 4/5 | ++ | +++ | ½ | Venteropostero Nuclei | 0 | + | 0 |
| Layer 6a/6b | +++ | 0 | + | MIDBRAIN | | | |
| Subiculum | ++ | ½ | ++ | Superior Colliculus | ½ | 0 | ½ |
| HIPPOCAMPUS | | | | APTD | ++ | ½ | ½ |
| CA1 | ++++ | +++ | ++++++ | DpMe | ½ | 0 | 0 |
| CA2 | ++++++ | +++ | ++++++ | Oculomotor Nucleus | + | 0 | ½ |
| CA3 | ++++++ | +++++ | ++++++ | Red Nucleus | + | ½ | 0 |
| Dentate Gyrus | +++++ | +++ | ++++++ | Interpeduncular Nucleus | + | 0 | 0 |
| Hilus Dentate Gyrus | ++ | + | ++ | Inferior Colliculus | | ½ | ½ |
| Indusium Gresium | ++++ | 0 | + | Central Grey | | 0 | ½ |
| Tenia Tecta | ++++ | + | ++++++ | BRAINSTEM | | | |
| BASAL GANGLIA | | | | Pontine Nucleus | 0 | +++ | 0 |
| Caudate Putamen | + | 0 | +++ | Trapezoid Body | + | ++ | 0 |
| Globus Palidus | 0 | 0 | 0 | Inferior Olivary Nucleus | 0 | + | ½ |
| Nucleus Accumbens | + | 0 | ++ | Locus Ceruleus | + | 0 | + |
| VBD | ½ | 0 | ½ | Raphe Nuclei | 0 | ½ | 0 |
| Habenula | ++++ | 0 | +++++ | Pontine Reticular Formation | 0 | ½ | 0 |
| Amygdala | + | 0 | ++ | Motor Trigeminal Nucleus | 1//2 | + | 0 |
| Hypothalamus | ½ | 0 | ½ | Me5 Cells | + | 0 | 0 |
| Preoptic Area | ½ | 0 | ½ | Facial Nuclei | 0 | ½ | 0 |
| Supraoptic | + | 0 | ½ | Vestibular Nuclei | 0 | ½ | 0 |
| Mammilary Body | + | 0 | + | Solitary Nucleus | 0 | ½ | 0 |
| Substantia Nigra | ½ | 0 | ½ | Cuneate Nucleus | 0 | +++ | 0 |
| VTA | | + | 0 | Dorsal Tegmental Nucleus | + | ½ | + |
| CEREBELLUM | | | | Lateral Parabrachial Nucleus | + | 0 | 0 |
| Granular Cell Layer | ++++++ | +++++ | 0 | Hypoglosseal Nucleus | ½ | 0 | 0 |
| Purkinje Cell Layer | +++ | +++++ | 0 | Spinal Trigeminal Nucleus | ½ | ½ | 0 |
| Molecular Cell Layer | 0 | 0 | 0 | SEPTUM | | | |
| | | | | Bed Nucleus Stria Terminalis | + | 0 | + |
| | | | | Lateral Septal Nucleus | + | 0 | + |

### Example 5

### Sequence Comparison and Three Dimensional Modelling of the Extracellular Domain of the β3 Sub Unit

Amino acid sequences of rat and human β3 were aligned with the sequences of rat β1 (SWISS-PROT Q00954) (Isom, L. L 1992); and the extracellular domain of rat myelin P₀ (SWISS-PROT P06907) (Lenike G. & Axel, R. 1985). The multiple alignment was generated with CLUSTALW (Higgins, D. G 1996) and formatted with ALSCRIPT (Barton, G. J. 1993) (FIG. 4). The sequence numbering is based on rat β3, starting from the predicted N-terminus of the mature protein. Amino acid identities with rat β3 are indicated by shading. The putative signal sequence and internalization signal are underlined and labelled. The putative transmembrane domain (TM) is boxed. Three negatively charged amino acid residues, previously identified as part of the α-sub-unit binding site of β1, are boxed. Invariant residues and the position of amino acids characteristic of the IgV domain are indicated beneath the sequence of myelin P₀: h, hydrophobic; 1, aliphatic; %, neutral or hydrophobic; +, base; =, hydrophobic or Ser, Thr; #, Gly or Ala (rarely Asp) (17). Secondary structure elements in the crystal structure of myelin P₀ (Shapiro, L 1996) used to model β3 are also shown: arrow, beta-strand; cylinder, alpha- or 3₁₀-helix.

The model for the three-dimensional structure of the mature extracellular domain (residues 1-123) of rat β3. The model was generated with MODELLER (Sali, A. & Blundell, T. L. 1993) using the crystal structure of rat myelin P₀ (PDB 1neu) (Shapiro, L 1996) as a template and the alignment shown in (FIG. 5). FIG. 5 was drawn with MOLSCRIPT (Kraulis, P. J. 1991) and RASTER3D (Merrit, E. & Murphy, M. 1994). The side chains of acidic residues in the putative α subunit binding site are shown in ball-and-stick representation. Two predicted disulfide bonds are labelled in black. N-linked glycosylation sites (NXT and NXS) (Kornfeld, R. & Kornfeld, S. 1985) are indicated by asterisks. The potential glycosylation site on the F strand (N97) points away from the viewer and is below the plane of the paper.

Note: In this model the B strand is broken into two parts labelled B and B′ respectively. This secondary structure assignment is based on the definition of Kabsch & Sander (1983) for the PDB entry 1neu and is different from the assignment described in the original paper (Shapiro, L 1996).

### Example 6

### Functional Expression of β3 Sub-Unit in a Recombinant System

Capped cRNA for rat brain type IIA α sub-unit and rat β3 sub-unit were transcribed in vitro from transcribed cDNAs (Promega, Southampton, UK). pBSK β3 was linearized with NotI and transcribed with T7 polymerase, while ZEMRVSP6-2580 α2 was linearized with ClaI and transcribed with SP6 polymerase. Xenopus laevis were anaesthetised by immersion in 0.3% (w/v) 3-amino benzoic acid (Sigma, Poole, U K) and Ovarian lobes were removed. Oocytes were dissociated using 0.3% (w/v) collagenase (Sigma, Poole, U K) in Ca²⁺-free solution (82.5 mM NaCl, 2.5 mM KCl, 1 mM MgCl₂, 5 mM, Hepes, pH 7.6). Prepared oocytes were microinjected with 50 nl of cRNAs dissolved in water. Oocytes were incubated at 18° C. in ND96 (96 mM NaCl; 2 mM KCl, 1.8 mM CaCl₂, 1 mM MgCl₂, 5 mM Hepes, pH 7.6). Two-electrode voltage clamp recordings were performed 3-6 days after microinjection of cRNAs using a Gene Clamp 500 amplifier (Axon Instruments, CA, USA) interfaced to a Digidata 1200 A/D board with Clampex software (v6, Axon Instruments, CA, USA). Oocytes were continually perfused with ND96, Microelectrodes filled with 3 M KCl had resistances between 0.5-2 MΩ. Currents were sampled at 10 kHz and filtered at 2 kHz. Data were analyzed using Clampfit (v6, Axon Instruments, CA, USA) and Prism (v2, Graphpad Software, CA, USA).

Inward Na⁺ currents were induced by applying 5 mV depolarizing pulses from a holding potential of −100 mV, from −80 mV to +30 mV. Na⁺ currents recorded from oocytes expressing IIA α subunit alone. Inactivation at −10 mV was best-fitted with a double exponential function, where τ₁=2±0.3 ms and τ₂=12.7±2.4 ms (n=4). b. Na⁺ currents recorded from oocytes coexpressing IIA α and β₁ subunits. Inactivation was best-fitted with a double exponential function, where τ₁=1.3±0.3 ms and τ₂=22.7±7.7 ms at −10 mV (n=4). c. Na⁺ currents recorded from oocytes coexpressing IIA α and the β₃ subunits. Inactivation was best-fitted with a double exponential function, where τ₁=1±0.1 ms and τ₂=23.8±6.3 ms at −10 mV (n=4).

Comparison of steady-state inactivation curves for Na⁺ currents recorded from oocytes coexpressing α2 and β3 with oocytes expressing }α2 alone. Data were fitted to Boltzmann function, g/gₘₐₓ=J/{1+exp[(V−V_{1/2})/k]}, where V_{1/2} is the midpoint and k is the slope factor. For α2+β3, V_{1/2}=−49.4 mV, k=10.1 mV, while for α2, V_{1/2}=−41.3 mV, k=9.1 mV. Co-expression of β3 with α2 causes a hyperpolarizing shift in the steady-state inactivation curve.

Comparison of rate of recovery from inactivation for Na⁺ currents recorded from oocytes co-expressing α2 and β3 with oocytes expressing α2 alone. Data were fitted with double exponential functions. For α2+β3, τ1=1.9 ms, τ2=198 ms while for α2, τ1=3.8 ms, τ2=264 ms. Co-expression of β3 with α2 increases the rate of recovery from inactivation.

The same procedures were used to express the human form of β3 and to measure the effects of co-expression with the type IIA α. For type IIA α alone Inactivation was best-fitted with a double exponential function, where τ₁=1.78±0.4 ms and τ₂=13±1.25 (n=4). For type IIA α and human β3, inactivation was best-fitted with a double exponential function, where τ₁=1±0.1 ms and τ₂=9.1±1.4 ms. t1/(t1+t2)=0.72+0.03.

This methodology is a technology for detecting changes in the function of the sodium channel complex as shown in FIG. 6.

### Example 7

### Functional Expression of β3 Sub-Unit in a Recombinant System

Capped cRNA for rat brain αIIA subunit and rat β1- or β3-subunits were transcribed in vitro from linearized cDNAs (Promega). Xenopus laevis were anaesthetised by immersion in 0.3% (w/v) 3-amino benzoic acid (Sigma) and ovarian lobes were removed. Oocytes were dissociated using 0.3% (w/v) collagenase (Sigma) in Ca²⁺-free solution (82.5 mM NaCl, 2.5 mM KCl, 1 mM MgCl₂, 5 mM, Hepes, pH 7.6). Prepared oocytes were microinjected with 50 nl of cRNAs (0.2-1 ng αcRNA, 10 ng of β1 or β3 cRNA) dissolved in water. The cRNA concentration was estimated by UV spectrophotometry and agarose gel electrophoresis. Oocytes were incubated at 18° C. in ND96 (96 mM NaCl, 2 mM KCl, 1.8 mM CaCl₂, 1 mM MgCl₂, 5 mM Hepes, pH 7.6). Two-electrode voltage clamp recordings were performed 3-6 days after microinjection of cRNAs using a GeneClamp 500 amplifier (Axon Instruments, Foster City, Calif., USA) interfaced to a Digidata 1200 A/D board with CLAMPEX software (version 6, Axon Instruments). Oocytes were continually perfused with ND96. Microelectrodes filled with 3 M KCl had resistances between 0.5-2 MΩ. Currents were sampled at 10 kHz and filtered at 2 kHz. Data were analysed using CLAMPFIT (version 6, Axon Instruments) and ORIGIN (version 5, Microcal Software, Northampton, Mass.). Exponential functions were fitted to data using the simplex fitting algorithm in CLAMPFIT.

(a) Na⁺ currents recorded from oocytes expressing rat αIIA, rat αIIA+rat β1 and rat αIIA+rat β3 subunits. Inward Na⁺ currents were evoked by applying depolarising pulses in 5 mV increments from a holding potential of −100 mV, from −80 mV to +30 mV. Duration of the pulses was 50 ms.

(b) Normalized Na⁺ currents from oocytes expressing rat αIIA, rat αIIA+rat β1 and rat αIIA+rat β3 subunits. Currents evoked by a voltage pulse to −10 mV were normalized to peak amplitudes. Inactivation of Na⁺ currents at −10 mV were fitted with a double exponential decay:

I=A1 exp(−t/τ1)+A2 exp(−t/τ2)+C, Where A1 and A2 are the relative amplitudes of fast and slow components τ1 and τ2 are the inactivation time constants and C is the steady-state asymptote. See table 2 for fit parameters.

(c). Recovery from inactivation of αIIA co-expressed with β1 or β3.

The recovery pulse protocol was a 1 s inactivating pulse to −10 mV followed by conditioning pulses to −100 mV for increasing periods of time (from 1-1000 ms), followed by a test pulse to −10 mV. Points were sampled every 1 ms from 1 to 20 ms, then every 50 ms from 50 to 1000 ms. Peak current amplitudes measured during the test pulse were normalized to the peak currents evoked during the inactivating pulse and were plotted as function of conditioning pulse duration. αIIA, αIIA+β1:, αIIA+β3: Data were fitted with a double exponential equation: I=1−[A1 exp (−t/τ1)+A2 exp (−t/τ2)] where A1 and A2 are the relative amplitudes of recovery and τ1 and τ2 are the recovery time constants. See table 2 for fit parameters. (d). Voltage-dependence of inactivation of αIIA co-expressed with β1 or β3. A two step protocol was applied using a conditioning pulse of 500 ms duration from −110 mV to +10 mV in 5 mV increments, followed by a test pulse to −10 mV. Peak current amplitudes evoked by the test pulse were normalized to the maximum peak current amplitude and plotted as a function of the conditioning pulse potential. Data were fitted with a two-state Boltzman equation: g=1/[1+exp{(V−V_{1/2})/k}], where g is conductance, V_{1/2} is the voltage of half-maximal inactivation and k is the slope factor. See table 2 for fit parameters.

TABLE 2

| | | | | | | | | Voltage-dependence | | | |
|---|---|---|---|---|---|---|---|---|---|---|---|
| Inactivation timecourse^{#} | | | | Recovery from inactivation^{#} | | | | of inactivation^{$} | | | |
| | | | Percentage | | | | Percentage | | | | |
| Subunit | τ1 (ms) | τ2 (ms) | in fast mode | n | τ1 (ms) | τ2 (ms) | in fast mode | n | V_{½}(mV) | k (mV) | n |
| αIIA | 2.4 ± 0.3 | 10.9 ± 1.3 | 46 ± 4 | 8 | 3.9 ± 0.2 | 446 ± 9 | 38 ± 0.4 | 5 | −45.1 ± 0.3 | 9.8 ± 0.2 | 4 |
| αIIA + β1 | 1.4 ± 0.2 | 24.7 ± 4.3 | 95 ± 1 | 5 | 2 ± 0.1 | 148 ± 19 | 84 ± 0.4 | 5 | −49.1 ± 0.3 | 5.9 ± 0.2 | 4 |
| αIIA + β3 | 1.5 ± 0.2 | 24.7 ± 4.3 | 85 ± 1 | 6 | 4.1 ± 0.2 | 170 ± 17 | 73 ± 1.2 | 5 | −49.3 ± 0.2 | 6.1 ± 0.1 | 4 |

### REFERENCES

Alting-Mees M A and Short J. M. 1994, Strategies 7 70-72 (E. coli XPORT/XLOLR helper phage RE704 system).

Ausubel, F. R., Brent, R., Kingston, R. E., Moore, D. D., Seidman, J. G., Smith, J. A. & Struhl, K. (1989) Curr. Protocols Mol. Biol. New York: Green Publishing Associates.

Avid H. and Leder P., (1972) Proc. Natl. Acad. Sci., 69:1408-1412.

Barton, G. J. (1993) Protein Eng. 6, 37-40.

Beaucage et al., Tetrahedron Lett (1981) 22: 1859-1862.

Bullock W O et al., 1987 Biotechniques 5; 376-379: (E. coli XLI Blue).

Brown El., Belagaje R, Ryan M J, Khorana H G, Methods Enzymol (1979); 68: 109-151.

Catterall, W. A. Yeasty Brew yields novel calcium channel inhibitor Nature Biotec 16 1998 906.

Chen et al., (1987) Mol. Cell. Biol., 7:2745-2752.

Chomczynski P and Saachi N. 1987 Single-step method of RNA isolation by acid guanidinium thiocyanate-phenol-chloroform extraction. Anal. Biochem 162; 156-159.

Diatchenko L. et al., 1996. PNAS 93: 6025-6030.

Eubanks, J., Srinvasan, J., dinulos, M. B., disteche, C. M. & Catterall, W. A. (1997) NeuroReport 8, 2775-2779.

Fraley et al. (1979), Proc. Natl. Acad. Sci. USA, 76:3348-3352.

Feldman and Steg, (1996) Medecine/Sciences, synthese, 12:47:55.

Flotte et al., (1992) Am. J. Respir. Cell Mol. Biol., 7:349:356.

Fields and Song, (1989) Nature, 340:245-246.

Graham et al., (1973) Virology, 52: 456-457.

Green et al., Ann. Rev. Biochem. 55:569-597 (1986).

Gopal (1985), Mol. Cell. Biol., 5:1188-1190.

Hames. B. D. Higgins, S. J. (1993). Transcription and translation (Practical approach series, ISBN 100190) Imprint Oxford: IRL Press, 1993.

Harland et al., (1985) J. Cell. Biol., 101: 1094-1095.

Harpaz. Y. & Chothia. C. (1994) J. Mol. Biol. 238, 528-39.

Higgins, D. G., Thompson, J. D. & Gibson, T. J. (1996) Methods Enzymol. 266, 383-402.

Houbenweyl, (1974), in Meuthode der Organischen Chemie, E. Wunsch Ed., Volume 15-I et 15-11, Thieme, Stuttgart.

Huygen et al., (1996) Nature Medicine, 2(8):893-898.

Huynh T V, Young R A, Davis R W. In DNA cloning (D M Glover, ed). Vol. 1, pp 56-110. IRL Press, Oxford, England 1985 (E. coli C600hfl).

Isom, L. L., De Jongh, K. & Catterall, W. A. (1994) Neuron 12, 1183-1194.

Isom, L. L., De Jongh, K., Patton, D., Reber, B., Offord, J., Charbonneau, H., Walsh, K., Goldin, A. L & Catterall, W. (1992) Science 256, 839-842.

Isom, L. L., Ragsdale, D., De Jongh, K., Westenbroek, R., Reber, B., Scheuer, T. & Catterall, W. (1995) Cell. 83, 433-442.

Isom, L. L. & Catterall, W. A. (1996) Nature (London) 383, 307-308.

Izant J G, Weintraub G, Cell 1984 April; 36(4):1007-15.

James, W. & Agnew, W. (1989). Alpha-(2-8)-polysialic acid immunoreactivity in voltage-sensitive sodium channel of eel electric organ. Proceedings of the Royal Society B 237, 233-245.

Julan et al., (1992) J. Gen. Virol., 73: 3251-3255.

Kabsch, W. & Sander, C. (1983) Biopolymers 22, 2577-2637.

Koch Y. (1977), Biochem. Biophys. Res. Commun., 74: 488-491.

Kohler G. and Milstein C., (1975) Nature, 256:495.

Kornfeld, R. & Kornfeld, S. (1985) A. Rev. Biochem. 54, 631-664.

Kozbor et al., (1983) Hybridoma, 2(1):7-16.

Kraulis, P. J. (1991) J. Appl. Crystallogr. 24, 946-950.

Leger O J, et al. (1997) Hum Antibodies, 8(1):3-16.

Lemke G. & Axel, R. (1985) Cell 40, 501-508.

Makielski; J. Limberis, J., Chang S., Fan, Z & Kyle, J. (1996). Molecular Pharmcology 49, 30-39.

Marks, M. S., Ohno, H., Kirchhausen, T. & Bonifacino, J. S. (1997) Trends Cell Biol. 7, 124-128.

Martineau P, Jones P, Winter G. (1998), J. Mol Biol, 280(1):117-127.

McCormick, K. A., Isom, L. L., Ragsdale, D., Smith, D., Scheuer, T. & Catterall, W. A. (1998) J. Biol. Chem. 273, 3954-3962.

McLaughlin B A et al., (1996), Am. J. Hum. Genet, 59:561-569.

Mead D A et al. Bio/Technology 9:657-633:1994 (PT-ADV plasmid).

Merrifield R B, 1965a, Nature, 207(996):522-523.

Merrifield R B, 1965b, Nature, 207 (996):522-523.

Merrit, E. & Murphy, M. (1994) Acta. Crystallogr. D. 50, 869-873.

Nada S. et al., (1993), Cell. 73:1125-1135.

Narang S A, Hsiung H M, Brousseau R, Methods Enzymol 1979: 68: 90-98.

Nguyen, C., Mattei, M-G., Mattei, J-F., Santoni, M-J., goridis, C., & Jordan, B. R. (1986) J. Cell Biol. 102, 711-715.

Nicolau C. et al., (1987), Methods Enzymol., 149:157-76.

Ohno et al., (1994), Science, 265:781-784.

O'Reilly et al., (1992) Baculovirus expression vectors: a Laboratory Manual. W.H. Freeman and Co., New York.

Patton, D., West. J. Catterall, W. & Goldin, A. (1992). Proceedings of the National Academy of Sciences of the USA 89, 10905-10909.

Potter et al., (1984) Proc Natl Acad Sci USA: 81-22):7161-5.

Qu, Y., Isom, L., Westenbroek, R. Rogers, J. Tanada, T., McCormick, K., Scheuer, T. & Catterall, W./ (1995). Modulation of cardia Na⁺ channel expression in Xenopus oocytes by beta 1-sub-units. Journal of Biological Chemistry 270, 25696-25701.

Tacson et al., (1996) Nature Medicine, 2(8):888-892.

Tur-Kaspa et al., (1986), Mol. Cell. Biol., 6:716-718.

Ridder R. Schmitz R, Legay F, Grain H, (1995) Biotechnology (NY), 13(3):255-260.

Rossi et al. Pharmacol. Ther. 50:245-254 (1991).

Roth J. A. et al., (1996) Nature Medicine, 2(9):985-991.

Rougeot, c. et al., (1994), Eur. J. Biochem., 219(3):765-773.

Roux et al., (1989) Proc. Natl. Acad. Sci. USA, 86:9079-9083.

Russell D W. Hirata R K 1998. Human gene targeting by viral vectors Nature Genetics. 18(4):325-330.

Sali, A. & Blundell, T. L. (1993) J. Mol. Biol. 234, 779-815.

Sambrook, J. Fritsch, E. F. and T. Maniatis (1989). Molecular cloning: a laboratory manual, 2ed. Cold Spring Harbor Laboratory, Cold Spring Harbor, N.Y.

Samulski et al., (1989) J. Virol. 63: 3822-3828.

Sanchez-Pescador R., (1988), J. Clin. Microbiol., 26(10): 1934-1938.

Sczakiel G. et al., (1995) Trends Microbiol., 3(6): 213-217.

Shapiro, L., Doyle, J. P., Hensley, P., Colman, D. R. & Hendrickson, W. A. (1996) Neuron 17, 435-49.

Shena et al., Science 270: 467-470, 1995.

Shena et al., 1996 Proc Natl Acad Sci USA. 93(20):10614-10619.

Short J M, Fernandez J M, Sarge J A, Huse W D 1988. NAR 16-7583-7600.

Smith et al., (1983), Mol. Cell. Biol., 3:2156-2165.

Sternberg N. L. (1992), Trends Genet, 8: 1-16.

Sternberg N. L. (1994) Mamm. Genome, 5:397-404.

Tacson et al., (1996) Nature Medicine, 2(8):888-1190

Urdea et al., MS (1988) Nucleic Acids Research, 11: 4937-4957.

Urdea et al., MS (1991) Nucleic Acids Symp Ser., 24: 197-200.

Von Heijne, G. (1983) Eur. J. Biochem. 133, 17-21.

Wallace, R. H., et al. (1998) Nature Gen. 19, 366-370.

Young, K. et al. Nature Biotech 16 1998 946-950.

Zhang Z. & Galilco, D. S. (1998). Neuroscience 18:6928-6938

The invention shall be further described in the following numbered paragraphs:

1. A purified or isolated nucleic acid encoding a β3 sub-unit from a voltage-gated sodium channel, or a sequence complementary thereto.

2. The nucleic acid of paragraph 1, which encodes a β3 sub-unit from the voltage-gated sodium channel present in the rat brain, or a sequence complementary thereto.

3. The nucleic acid of paragraph 1, which encodes the β3 sub-unit from the voltage-gated sodium channel present in the human brain, or a sequence complementary thereto.

4. A purified or isolated nucleic acid according to paragraph 1, wherein said nucleic acid encodes a polypeptide having at least 80% amino acid identity with the β3 sub-unit polypeptide of the amino acid sequence of SEQ ID NO 1, or with a peptide fragment thereof, or a sequence complementary thereto.

5. A purified or isolated nucleic acid according to paragraph 1, wherein said nucleic acid encodes a polypeptide having at least 80% amino acid identity with the β3 sub-unit polypeptide of the amino acid sequence of SEQ ID NO 2, or a sequence complementary thereto.

6. A purified or isolated nucleic acid according to paragraph 1, wherein said nucleic acid has at least 90% nucleotide identity with the nucleotide sequence of SEQ ID NO 3, or a sequence complementary thereto.

7. A purified or isolated nucleic acid according to paragraph 1, wherein said nucleic acid comprises a polynucleotide having at least 90% nucleotide identity with the sequence beginning at the nucleotide located in position 363 and ending at the nucleotide located in position 1010 of the nucleotide sequence of SEQ ID N°3.

8. A purified or isolated nucleic acid according to paragraph 1, wherein said nucleic acid comprises a sequence beginning at the nucleotide located in position 1 and ending at the nucleotide located in position 362 of the nucleotide sequence of SEQ ID N°3.

9. A purified or isolated nucleic acid according to paragraph 1, wherein said nucleic acid comprises a sequence beginning at the nucleotide located in position 1011 and ending at the nucleotide located in position 2220 of the nucleotide sequence of SEQ ID N°3.

10. A purified or isolated nucleic acid according to paragraph 1, wherein said nucleic acid has at least 90% nucleotide identity with the nucleotide sequence of SEQ ID NO 4, or a sequence complementary thereto.

11. A purified or isolated nucleic acid according to paragraph 1, wherein said nucleic acid comprises a polynucleotide having at least 90% nucleotide identity with the sequence beginning at the nucleotide located in position 376 and ending at the nucleotide in position 1023 of the nucleotide sequence of SEQ ID N°4.

12. A purified or isolated nucleic acid according to paragraph 1, wherein said nucleic acid comprises a sequence beginning at the nucleotide located in position 1 and ending at the nucleotide located in position 375 of the nucleotide sequence of SEQ ID N°4.

13. A purified or isolated nucleic acid according to paragraph 1, wherein said nucleic acid comprises a sequence beginning at the nucleotide located in position 1024 and ending at the nucleotide located in position 1261 of the nucleotide sequence of SEQ ID N°4.

14. A purified or isolated polynucleotide comprising at least 10 consecutive nucleotides of a nucleic acid encoding a β3 sub-unit of a voltage-gated sodium channel.

15. A purified or isolated nucleic acid according to paragraph 14, wherein said nucleic acid comprises at least 10 consecutive nucleotides of the nucleotide sequence of SEQ ID NO 3, or a sequence complementary thereto.

16. A purified or isolated nucleic acid according to paragraph 14, wherein said nucleic acid comprises at least 10 consecutive nucleotides of the nucleotide sequence of SEQ ID NO 4, or a sequence complementary thereto.

17. A purified or isolated nucleic acid according to paragraph 14, wherein said nucleic acid is selected from the group consisting of SEQ ID N° 35 to 43 or a polynucleotide encoding a peptide of SEQ ID N° 5 to 32, SEQ ID N° 46 or SEQ ID No 47.

18. A method for the amplification of a β3 subunit nucleic acid, said method comprising the steps of:

a) contacting a test sample suspected of containing the targeted β3 subunit nucleic acid or a fragment thereof with amplification reaction reagents comprising a pair of amplification primers which can hybridize to a nucleic acid according to any one of paragraphs 1 to 17, and

b) optionally, detecting the amplification products.

19. The method according to paragraph 18, wherein the amplification primers are respectively the nucleotide sequences of SEQ ID Nos 33 and 35.

20. A kit for the amplification of a β3 subunit nucleotide sequence, wherein said kit comprises:

a) a pair of amplification primers which can hybridize to a β3 subunit nucleic acid according to any one of paragraphs 1 to 17, and

b) optionally, the reagents necessary for performing the amplification reaction.

21. A method for detecting the presence of polynucleotide comprising a nucleic acid according to any one of paragraphs 1 to 17 in a sample, wherein said method comprises the steps of:

a) bringing into contact a nucleic acid probe or a plurality of nucleic acid probes which can hybridize, under stringent hybridization conditions, to a nucleotide sequence included in a nucleic acid according to any one of paragraphs 1 to 17, and the sample to be assayed;

b) detecting the hybrid complex formed between the probe or the plurality of probes and the nucleic acid in the sample.

22. The method of paragraph 21, wherein the nucleic acid probe or the plurality of nucleic acid probes are immobilized on a substrate.

23. The method of paragraph 21, wherein the nucleic acid probe or the plurality of nucleic acid probes is labeled with a detectable molecule.

24. A kit for detecting the presence of a polynucleotide comprising a nucleic acid according to any one of paragraphs 1 to 17, wherein said kit comprises:

a) a nucleic acid probe or a plurality of nucleic acid probes which can hybridize, under stringent hybridization conditions, to a nucleotide sequence included in a nucleic acid according to any one of paragraphs 1 to 16;

b) optionally, the reagents necessary to perform the hybridization reaction.

25. The kit of paragraph 24, wherein the nucleic acid probe or the plurality of nucleic acid probes are immobilized on a substrate.

26. The kit of paragraph 24, wherein the nucleic acid probe or the plurality of nucleic acid probes are labeled with a detectable molecule.

27. A recombinant vector comprising a nucleic acid according to any one of paragraphs 1 to 17.

28. A recombinant host cell comprising a nucleic acid according to any one of paragraphs 1 to 17.

29. A method for producing a polypeptide encoded by a nucleic acid according to any one of paragraphs 1 to 7, 10, 11 and, 14 to 17, wherein said method comprises the following steps of:

a) culturing, in an appropriate culture medium, a host cell previously transformed or transfected with a polynucleotide according to any one of paragraphs 1 to 7, 10, 11 and, 14 to 17;

b) harvesting the culture medium thus conditioned or lyse the host cell, for example by sonication or by osmotic shock; and

c) separating or purifying, from said culture medium, or from the pellet of the resulting cell lysate, the thus produced polypeptide of interest.

30. A purified or isolated polypeptide comprising the amino acid sequence of the β3 sub-unit from a voltage-gated sodium channel, or a peptide fragment thereof.

31. The polypeptide of paragraph 30, which comprises the amino acid sequence of the β3 sub-unit from a voltage-gated sodium channel present in the rat brain, or a peptide fragment thereof.

32. The polypeptide of paragraph 30, which comprises the amino acid sequence of the β3 sub-unit from a voltage-gated sodium channel present in the human brain, or a peptide fragment thereof.

33. A purified or isolated polypeptide comprising an amino acid sequence having at least 90% amino acid identity with the amino acid sequence of SEQ ID NO 1, or a peptide fragment thereof.

34. A purified or isolated polypeptide comprising an amino acid sequence having at least 90% amino acid identity with the amino acid sequence of SEQ ID NO 2, or a peptide fragment thereof.

35. A purified or isolated polypeptide encoded by a nucleic acid of any one of paragraphs 1 to 7, 10, 11, 14 to 17.

36. A purified or isolated polypeptide selected from the group consisting of the polypeptides of SEQ ID N° 5 to 32 and SEQ ID 46 and 47.

37. A method for screening ligand substances or molecules that are able to modulate the biological activity of a voltage-gated sodium channel containing a β3 sub-unit, said method comprising:

(a) obtaining a recombinant host cell co-expressing a β3 sub-unit or a fragment thereof and a functional α sub-unit, preferably an α2 sub-unit of a voltage-gated sodium channel, or a fragment thereof,

(b) bringing into contact said recombinant host cell with a substance or molecule to be tested; and

(c) measuring an electrical parameter within the recombinant host cell brought into contact with the substance or molecule to be tested through a voltage clamp technique or measurement of membrane potential by voltage sensitive fluorescent dyes.

38. A method for screening ligand substances or molecules that are able to modulate the biological activity of a voltage-gated sodium channel containing a β3 sub-unit, said method comprising:

(a) contacting the ligand with the β3 sub-unit or a fragment thereof;

(b) contacting the medium containing the ligand and the β3 protein or a fragment thereof with a β3 substrate and allowing the possible binding of the substrate to the β3 protein or a fragment thereof to occur; and

(c) measuring the eventual binding of the substrate to the β3 protein or a fragment thereof.

What is claimed is:

1. A purified or isolated nucleic acid encoding a β3 sub-unit from a voltage-gated sodium channel, or a sequence complementary thereto.

2. The nucleic acid of claim 1, which encodes β3 sub-unit from the voltage-gated sodium channel present in the rat brain, or a sequence complementary thereto.

3. The nucleic acid of claim 1, which encodes the β3 sub-unit from the voltage-gated sodium channel present in the human brain, or a sequence complementary thereto.

4. A purified or isolated nucleic acid according to claim 1, wherein said nucleic acid encodes a polypeptide having at last 80% amino acid identity with the β3 sub-unit polypeptide of the amino acid sequence of SEQ ID NO 1 over the entire length of the sequence of SEQ ID NO 1, or with a peptide fragment thereof, or a sequence complementary thereto, with the exception of the nucleic acid of EMMETT database having accession NO AA685538.

5. A purified or isolated nucleic acid according to claim 1, wherein said nucleic acid encodes a polypeptide having a least 80% amino acid identity with the β3 sub-unit polypeptide of the amino acid sequence of SEQ ID NO 2 over the entire length of the sequence of SEQ ID NO 2, or a sequence complementary thereto.

6. A purified or isolated nucleic acid according to claim 1, wherein said nucleic acid has at least 90% nucleotide identity with the nucleotide sequence of SEQ ID NO 3 over the entire length of the sequence of SEQ ID NO 3, or a sequence complementary thereto.

7. A purified or isolated nucleic acid according to claim 1, wherein said nucleic acid comprises a polynucleotide having at least 90% nucleotide identity with th sequence beginning at the nucleotide located in position 363 and ending at the nucleotide located in position 1010 of the nucleotide sequence of SEQ ID N°3.

8. A purified or isolated nucleic acid according to claim 1, wherein said nucleic acid comprises a sequence beginning at the nucleotide located in position 1 and ending at the nucleotide located in position 362 of the nucleotide sequence of SEQ ID N°3.

9. A purified or isolated nucleic acid according to claim 1, wherein said nucleic acid comprises a sequence beginning at the nucleotide located in position 1011 and ending at the nucleotide located in position 2220 of the nucleotide sequence of SEQ ID N°3.

10. A purified or isolated nucleic acid according to claim 1, wherein said nucleic acid has at least 90% nucleotide identity with the nucleotide sequence of SEQ ID NO 4 over the entire length of the sequence of SEQ ID NO 4, or a sequence complementary thereto.

11. A purified or isolated nucleic acid according to claim 1, wherein said nucleic acid comprises a polynucleotide having at least 90% nucleotide identity with the sequence beginning at the nucleotide located in position 376 and ending at the nucleotide in position 1023 of the nucleotide sequence of SEQ ID N°4.

12. A purified or isolated nucleic acid according to claim 1, wherein said nucleic acid comprises a sequence beginning at the nucleotide located in position 1 and ending at the nucleotide located in position 375 of the nucleotide sequence of SEQ ID N°4.

13. A purified or isolated nucleic acid according to claim 1, wherein said nucleic acid comprises a sequence beginning at the nucleotide located in position 1024 and ending at the nucleotide located in position 1261 of the nucleotide sequence of SEQ ID N°4.

14. A purified or isolated polynucleotide comprising at least 10 consecutive nucleotides of a nucleic acid encoding a β3 sub-unit of a voltage-gated sodium channel, with the exception of the polynucleotide bearing SEQ ID NO 876 in W09845435 and the polynucleotide of EMEST database having accession NO AA685538.

15. A purified or isolated nucleic acid according to claim 14, wherein said nucleic acid comprises at least 10 consecutive nucleotides of the nucleotide sequence of SEQ ID NO 3, or a sequence complementary thereto.

16. A purified or isolated nucleic acid according to claim 14, wherein said nucleic acid comprises at least 10 consecutive nucleotides of the nucleotide sequence of SEQ ID NO 4, or a sequence complementary thereto.

17. A purified or isolated nucleic acid according to claim 14, wherein said nucleic acid is selected from the group consisting of SEQ ID N° 35 to 43 or a polynucleotide encoding a peptide of SEQ ID N° 5 to 32, SEQ ID N° 46 or SEQ ID N° 47.

18. A method for the amplification of a β3 subunit nucleic acid, said method comprising the steps of
a) contacting a test sample suspected of containing the targeted β3 subunit nucleic acid or a fragment thereof with amplification reaction reagents comprising a pair of amplification primers which can hybridize to a nucleic acid according to any one claims 1 to 17, and
b) optionally, detecting the amplification products.

19. The method according to claim 18, wherein the amplification primers are respectively the nucleotide sequences of SEQ ID Nos 33 and 35.

20. A kit for the amplification of a β3 subunit nucleotide sequence, wherein said kit comprises
a) a pair of amplification primers which can hybridize to a β3 subunit nucleic acid according to any one of claims 1 to 17, and
b) optionally, the reagents necessary for performing the amplification reaction.

21. A method for detecting the presence of polynucleotide comprising a nucleic acid according to any one of claims 1 to 17 in a sample, wherein said method comprises the steps of
a) bringing into contact a nucleic acid probe or a plurality of nucleic acid probes which can hybridize, under stringent hybridization conditions, to a nucleotide sequence included in a nucleic acid according to any one of claims 1 to 17, and the sample to be assayed;
b) detecting the hybrid complex formed between the probe or the plurality of probes and the nucleic acid in the sample.

22. The method of claim 21, wherein the nucleic acid probe or the plurality of nucleic acid probes are immobilized on a substrate.

23. The method of claim 21, wherein the nucleic acid probe or the plurality of nucleic acid probes is labeled with a detectable molecule.

24. A kit for detecting the presence of a polynucleotide comprising a nucleic acid according to any one of claims 1 to 17, wherein said kit comprises
a) a nucleic acid probe or a plurality of nucleic acid probes which can hybridize, under stringent hybridization conditions, to a nucleotide sequence included in a nucleic acid according to any one of claims 1 to 16;
b) optionally, the reagents necessary to perform the hybridization reaction.

25. The kit of claim 24, wherein the nucleic acid probe or the plurality of nucleic acid probes are immobilized on a substrate.

26. The kit of claim 24, wherein the nucleic acid probe or the plurality of nucleic acid probes are labeled with a detectable molecule.

27. A recombinant vector comprising a nucleic acid according to any one of claims 1 to 17.

28. A recombinant host cell comprising a nucleic acid according to any one of claims 1 to 17.

29. A method for producing a polypeptide encoded by a nucleic acid according to any one of claims 1 to 7, 10, 11 and, 14 to 17, wherein said method comprises the following steps of
a) culturing, in an appropriate culture medium, a host cell previously transformed or transfected with a polynucleotide according to any one of claims 1 to 7, 10, 11 and, 14 to 17;
b) harvesting the culture medium thus conditioned or lyse the host cell, for example by sonication or by osmotic shock; and c) separating or purifying, from said culture medium, or from the pellet of the resulting cell lysate, the thus produced polypeptide of interest.

30. A purified or isolated polypeptide comprising the amino acid sequence of the β3 sub-unit from a voltage-gated sodium channel, or a peptide fragment thereof.

31. The polypeptide of claim 30, which comprises the amino acid sequence of the β3 sub-unit from a voltage-gated sodium channel present in the rat brain, or a peptide fragment thereof.

32. The polypeptide of claim 30, which comprises the amino acid sequence of the β3 sub-unit from a voltage-gated sodium channel present in the human brain, or a peptide fragment thereof.

33. A purified or isolated polypeptide comprising an amino acid sequence having at least 90% amino acid identity with the amino acid sequence of SEQ ID NO 1 over the entire length of the sequence of SEQ ID NO 1, or a peptide fragment thereof.

34. A purified or isolated polypeptide comprising an amino acid sequence having at least 90% amino acid identity with the amino acid sequence of SEQ ID NO 2 over the entire length of the sequence of SEQ ID NO 2, or a peptide fragment thereof.

35. A purified or isolated polypeptide encoded by a nucleic acid of any one of claims 1 to 7, 10, 11, 14 to 17.

36. A purified or isolated polypeptide selected from the group consisting of the polypeptides of SEQ ID N° 5 to 32 and SEQ ID 46 and 47.

37. A method for screening ligand substances or molecules that are able to modulate the biological activity of a voltage-gated sodium channel containing a β3 sub-unit, said method comprising:
(a) obtaining a recombinant host cell co-expressing a β3 sub-unit or a fragment thereof and a functional a sub-unit, preferably an α2 sub-unit of a voltage-gated sodium channel, or a fragment thereof;
(b) bringing into contact said recombinant host cell with a substance or molecule to be tested; and
(c) measuring an electrical parameter within the recombinant host cell brought into contact with the substance or molecule to be tested through a voltage clamp technique or measurement of membrane potential by voltage sensitive fluorescent dyes.

38. A method for screening ligand substances or molecules that are able to modulate the biological activity of a voltage-gated sodium channel containing a β3 sub-unit, said method comprising:
(a) contacting the ligand with the β3 sub-unit or a fragment thereof;
(b) contacting the medium containing the ligand and the β3 protein or a fragment thereof with a β3 substrate and allowing the possible binding of the substrate to the β3 protein or a fragment thereof to occur; and
(c) measuring the eventual binding of the substrate to the β3 protein or a fragment thereof.
